(19) United States
(12) Patent Application Publication
(10) Pub. No.: US 2020/0355685 A1
(43) Pub. Date: Nov. 12, 2020

(54) REAGENT SET FOR DETECTING INTERACTIONS BETWEEN BIOMOLECULES AND THEIR REGULATORY FACTORS, AND APPLICATIONS

(71) Applicant: TSINGHUA UNIVERSITY, Beijing (CN)

(72) Inventors: Pilong LI, Beijing (CN); Min ZHOU, Beijing (CN); Jing WANG, Beijing (CN); Jian LI, Beijing (CN); Weiping LI, Beijing (CN); Liang WANG, Beijing (CN); Rongbo WU, Beijing (CN)

(21) Appl. No.: 16/762,051

(22) PCT Filed: Nov. 1, 2018
(86) PCT No.: PCT/CN2018/113300

§ 371 Date: May 6, 2020

(30) Foreign Application Priority Data
Nov. 6, 2017 (CN) 201711079545.6
Dec. 12, 2017 (CN) 201711315673.6
Aug. 1, 2018 (CN) 201810862836.0

(51) Int. Cl. G01N 33/573

(52) U.S. Cl. CPC G01N 33/573; G01N 2333/9015; G01N 2333/91215

(57) ABSTRACT

The present invention discloses a reagent set for detecting interactions between biomolecules and their regulatory factors and applications. One reagent set disclosed in the present invention consists of three reagents named A, B and C; the reagent A is formed by connecting a biomolecule R and a biomolecule X; the reagent B contains a biomolecule L; there is an interaction between the biomolecule R and the biomolecule L, and a phase transition occurs when the biomolecule R and the biomolecule L interact; the reagent C is formed by connecting a reporter group JIA with a biomolecule named X_{L}. Another reagent set disclosed in the present invention consists of four reagents named A, B, E and D; the reagent E is a polymer formed by E monomers, and the E monomer is obtained by connecting a monomer mc, a reporter group JIA, and a biomolecule Y_{C}, two or more mc monomers can form a polymer; the reagent D is formed by connecting a modified protein X_{L} and a biomolecule Y_{D}; there is an interaction between the biomolecule Y_{C} and the biomolecule Y_{D}. The present invention can be used to detect the intracellular and extracellular protein interaction or even the weak interaction and has the characteristics of high visibility, simple operation, low cost, high sensitivity and wide applicability.

### RELATED APPLICATIONS

The present application is a National Phase of International Application Number PCT/CN2018/113300, filed Nov. 1, 2018, and claims the priority of China Application No. 201711079545.6, filed Nov. 6, 2017, and China Application No. 201711315673.6, filed Dec. 12, 2017, and China Application No. 201810862836.0, filed Aug. 1, 2018.

### INCORPORATION BY REFERENCE

The sequence listing provided in the file entitled C6351-024_SEQUENCE_LISTING_v2.txt, which is an ASCII text file that was created on Jul. 15, 2020, and which comprises 54,951 bytes, is hereby incorporated by reference in its entirety.

### TECHNICAL FIELD

The present invention relates to a reagent set for detecting interactions between biomolecules and their regulatory factors and applications in the field of biotechnology.

### BACKGROUND ART

As a property of matter, “phase transition” has been widely known in the field of physics and daily life. In recent years, scientists have discovered that the phase transition (or phase separation) mechanism also exists widely in biological cells, and plays an important role in spatiotemporal regulation of cell cycle.

Current research has found that when a multivalent macromolecule in solution interacts with its multivalent ligand, it is easy to produce a larger complex and the larger complex generally has a reduced solubility and thus separated from the ordinary solution phase to form a complex-rich, independent liquid phase (referred to as a second phase to distinguish it from the original solution phase). This transformation process is called “liquid-liquid phase separation”. In this transformation process, the valence of the multivalent molecule refers to the number of binding regions contained in the macromolecule or in its ligand that can interact with each other. For protein interactions, multivalent proteins and their multivalent ligands will also undergo a “liquid-liquid phase separation” (referred to as “phase transition” for simplicity) phenomenon in vitro, which can produce a normal solution phase and a protein-rich, viscous liquid phase (i.e., a second phase). It can be seen under the microscope that the protein-rich liquid phase contains a large number of small droplets (i.e., phase transition droplets), and the droplets can be as large as micron-scale or larger in diameter and have a relatively high recognition. For example, multivalent SH3 (SRC homology 3 domain) and its multivalent ligand PRM (proline-rich motif) can undergo a phase transition at a certain concentration, while PRMH, which has a higher affinity for SH3, can undergo a stronger phase transition with SH3.

In organisms, the interaction between a protein and its ligand is the main way a protein performs its function. Under physiological conditions, the interaction between proteins exists in a form of dynamic equilibrium, and the dissociation constant (Kd) is often used to characterize the strength of protein interactions. According to the different Kd values, protein interactions are generally divided into stable interaction and transient interaction. The former corresponds to a Kd value ranging from pM to μM, and the latter corresponds to a Kd value more than 1 μM. Basically, transient interactions between proteins can be considered as weak protein interactions. The interactions between modified proteins (including methylation/demethylation, acetylation/deacetylation, phosphorylation/dephosphorylation, ubiquitination/deubiquitination, glycosylation/deglycosylation, etc.) and their ligands are mostly weak interactions. This weak interaction plays an important role in cellular signal transduction and cell cycle regulation, etc. For example, the weak interaction between a phosphorylated protein and its ligand can realize the transfer of phosphate groups in the signal pathway, and the weak interaction between a methylated histone and its ligand can regulate the expression of genes. It indicates that studying the weak interactions between proteins helps to understand important cytological processes. However, currently the weak interactions between proteins are still difficult to detect.

In biological cells, the interaction between intrinsically disordered proteins/regions (referred to as IDPs/IDRs for short) is an important mechanism driving the phase transition. The intrinsically disordered proteins/regions refer to as a class of proteins/protein regions that have no stable and ordered secondary and/or tertiary structure under physiological conditions, and do not fold in whole or in part in the natural state, but can normally perform biological functions. They exist widely in organisms and play important roles in cellular signal transduction and protein interaction networks. The disordered domains usually have preference in amino acid composition, such as rich in polar amino acids such as G. P, E, S, Q, K, D, T, and R and aromatic amino acids such as Y and F. It was found that the N-terminus of nucleoporin NUP98 anchored to the nuclear pore complex contains IDRs which can mediate the occurrence of phase transitions.

### SUMMARY OF THE INVENTION

The technical problem to be solved by the present invention is how to detect interactions between biomolecules and how to detect the influence of regulatory factors on the interactions between biomolecules. A further technical problem to be solved is how to detect weak interactions between proteins, especially the interactions between post-translationally modified proteins and their ligands. Post-translational modification refers to the covalent modification process that occurs on specific amino acid residues of proteins after the protein translation process. At present, more than 300 post-translational modifications have been found and the common ones contain methylation, acetylation, phosphorylation, ubiquitination, glycosylation, etc. The modification process opposite to protein post-translational modification is protein de-modification, such as demethylation, deacetylation, dephosphorylation, deubiquitination, deglycosylation, etc.

In order to solve the above technical problems, the present invention first provides a reagent set for detecting interactions between biomolecules and detecting the influence of regulatory factors on the interactions between biomolecules. The reagent set is named reagent set 1, which consists of three reagents named A, B and C, respectively;

the reagent A is formed by connecting a biomolecule named R and a biomolecule named X;
the reagent B contains a biomolecule named L;
the biomolecule R and the biomolecule L are the same or different and there is an interaction between the two, and a phase transition occurs when the biomolecule R and the biomolecule L interact;
the reagent C is formed by connecting a reporter group named JIA with a biomolecule named X_{L};
the biomolecule X is a protein, a nucleic acid, or a polysaccharide; the biomolecule X_{L} is a protein, a nucleic acid, or a polysaccharide.

In the above reagent set 1, it is unknown whether there is an interaction between the biomolecule X and the biomolecule X_{L}, and the reagent set 1 can be used to detect or assist in detecting whether there is an interaction between the biomolecule X and the biomolecule X_{L}.

In the above reagent set 1, there is an interaction between the biomolecule X and the biomolecule X_{L}, and the reagent set 1 can be used to identify or assist in identifying a regulatory factor for the interaction between the biomolecule X and the biomolecule X_{L}.

The present invention further provides a reagent set for detecting whether there is an interaction between a protein named X and a modified protein named X_{L}, which is named reagent set 3 (i.e., the reagent set 1 in Chinese Patent Application No. 201711315673.6), and the reagent set 3 consists of four reagents named A, B, E (i.e., the reagent C in Chinese Patent Application No. 201711315673.6) and D, respectively;

the reagent A is formed by connecting a biomolecule named R and a biomolecule named X; the biomolecule X is a protein:
the reagent B contains a biomolecule named L:
the biomolecule R and the biomolecule L are the same or different and there is an interaction between the two, and a phase transition occurs when the biomolecule R and the biomolecule L interact;
the reagent E is a polymer formed by E monomers (i.e., the C monomers in Chinese Patent Application No. 201711315673.6), and the E monomer is the following c1) or c2):
c1) a molecule obtained by connecting a monomer named mc, a reporter group named JIA, and a biomolecule named Y_{C}, two or more mc monomers can form a polymer;
c2) a molecule obtained by ligating a tag to c1);
the reagent D is formed by connecting a modified protein named X_{L} and a biomolecule named Y_{D};
there is an interaction between the biomolecule Y_{C} and the biomolecule Y_{D}.

In the above reagent set 3, both the biomolecule Y_{C} and the biomolecule Y_{D} can be proteins.

In the above reagent set 3, the biomolecule Y_{C} can be the following Y11), Y12) or Y13):

Y11) a protein having the amino acid sequence as shown in positions 362-465 of SEQ ID NO: 19;
Y12) a protein obtained by substitution and/or deletion and/or addition of one or more amino acid residues in the amino acid sequence as shown in positions 362-465 of SEQ ID NO: 19 in the Sequence Listing and having the same function;
Y13) a fusion protein obtained by ligating tag(s) to the N-terminus or/and C-terminus of Y11) or Y12).

The biomolecule Y_{D} can be the following Y21), Y22) or Y23):

Y21) a protein having the amino acid sequence as shown in positions 22-29 of SEQ ID NO: 23:
Y22) a protein obtained by substitution and/or deletion and/or addition of one or more amino acid residues in the amino acid sequence as shown in positions 22-29 of SEQ ID NO: 23 in the Sequence Listing and having the same function;
Y23) a fusion protein obtained by ligating tag(s) to the N-terminus or/and C-terminus of Y21) or Y22).

Wherein, KKETPV as shown in positions 22-29 of SEQ ID NO: 23 interacts with PDZ as shown in positions 362-465 of SEQ ID NO: 19.

In the above reagent set 1 and reagent set 3, the biomolecule R contains a binding region named binding region 1; the biomolecule L contains a binding region named binding region 2; and the interaction between the biomolecule R and the biomolecule L is realized by the binding region 1 and the binding region 2, and both the number of the binding region 1 in the biomolecule R and the number of the binding region 2 in the biomolecule L can be greater than or equal to 2.

Wherein, the binding region 1 and the binding region 2 are both binding regions, and the binding region refers to the smallest unit of interaction between biomolecules through non-covalent bonds. When there are two or more binding regions between the biomolecule R and the biomolecule L, if the binding regions in the biomolecule R are not completely the same, all the binding regions in the biomolecule R are collectively referred to as the binding region 1, and if the binding regions in the biomolecule L are not completely the same, all the binding regions in the biomolecule L are collectively referred to as the binding region 2.

Both the biomolecule R and the biomolecule L are multivalent molecules. Wherein, the valence of a multivalent molecule refers to the number of binding regions contained in one molecule that can bind to another molecule when the molecules interact with each other. For the biomolecule R, the valence of the biomolecule R is the number of the binding region 1, and for the biomolecule L, the valence of the biomolecule L is the number of the binding region 2.

The biomolecule R and the biomolecule L undergo a phase transition through a multivalent interaction.

The reagent set 1 can detect interactions between proteins and proteins, nucleic acids and nucleic acids, proteins and nucleic acids, and proteins and polysaccharides.

In the above reagent set 1 and reagent set 3, the biomolecule R can be a protein, a nucleic acid, or a polysaccharide. The biomolecule L can be a protein, a nucleic acid, or a polysaccharide.

In the above reagent set 1 and reagent set 3, a reporter group named YI can be further connected to the reagent A.

A reporter group named BING can also be connected to the reagent B.

In the above reagent set 1 and reagent set 3, the reporter group YI and the reporter group BING are the same or different.

The reporter group JIA is different from the reporter group YI and the reporter group BING.

In the above reagent set 1 and reagent set 3, the reporter groups JIA, YI and BING can all be fluorescent reporter groups. Further, the reporter groups JIA, YI and BING can all be fluorescent proteins.

In the above reagent set 1 and reagent set 3, the ratio of the number of the biomolecule X to the number of the biomolecule R in the reagent A can be an integer greater than or equal to 1.

The molar ratio of the reporter group JIA to the biomolecule X_{L} in the reagent C can be 1:1.

In the reagent C, the reporter group JIA and the biomolecule X_{L} can be connected through a linking region or a chemical bond. The reporter group JIA can specifically be mCherry.

In the E monomer, the molar ratio of the monomer mc, the reporter group JIA and the biomolecule Y_{C} can be 1:1:1.

In the E monomer, the monomer mc, the reporter group JIA and the biomolecule Y_{C} can be connected through a linking region or a chemical bond. The reporter group JIA can specifically be mCherry.

In the above reagent set 1 and reagent set 3, the biomolecule R can be a polymer formed by R monomers, and each R monomer contains a monomer named mr, and two or more mr monomers can form a polymer;

the biomolecule L can be a polymer formed by L monomers, and each L monomer contains a monomer named ml, and two or more ml monomers can form a polymer:
the mc monomer, the mr monomer and the ml monomer are the same or at least two of them are the same or they are different from each other.

In the above reagent set 1 and reagent set 3, at least one monomer in the biomolecule R contains the binding region 1.

At least one monomer in the biomolecule L contains the binding region 2.

When only one monomer in the biomolecule R contains the binding region 1, the monomer contains at least two of the binding region 1, and when two or more monomers in the biomolecule R contain the binding region 1, the number of the binding region 1 in each monomer is at least one.

When only one monomer in the biomolecule L contains the binding region 2, the monomer contains at least two of the binding region 2, and when two or more monomers in the biomolecule L contain the binding region 2, the number of the binding region 2 in each monomer is at least one.

In the monomer containing the binding region 1 of the biomolecule R, the binding region 1 can be connected to the mr monomer.

In the monomer containing the binding region 2 of the biomolecule L, the binding region 2 can be connected to the ml monomer.

The mr monomer is the same as or different from the ml monomer.

In the above reagent set 1 and reagent set 3, each R monomer can contain the mr monomer and the binding region 1.

Each L monomer can contain the ml monomer and the binding region 2.

In the above reagent set 1 and reagent set 3, in the R monomer, the mr monomer and the binding region 1 or a biomolecule containing the binding region 1 can be connected through a linking region or a chemical bond.

In the L monomer, the ml monomer and the binding region 2 or a biomolecule containing the binding region 2 can be connected through a linking region or a chemical bond.

In the above reagent set 1 and reagent set 3, each R monomer can further contain the reporter group YI.

Each L monomer can further contain the reporter group BING.

In the above reagent set 1 and reagent set 3, in the R monomer, the mr monomer, the reporter group YI, and the binding region 1 or a biomolecule containing the binding region 1 can be connected through a linking region or a chemical bond.

In the L monomer, the ml monomer, the reporter group BING, and the binding region 2 or a biomolecule containing the binding region 2 can be connected through a linking region or a chemical bond.

In the R monomer, the number of the binding region 1 is at least one.

In the L monomer, the number of the binding region 2 is at least one.

In the R monomer and L monomer, no matter the number of each part (i.e., the mr monomer or the ml monomer, the binding region 1 or the binding region 2, or the reporter group YI or the reporter group BING) is 1 or more, there is no requirement for the order of connection between each other, as long as two or more R monomers can form a polymer, two or more L monomers can form a polymer, and these two polymers can interact and cause a phase transition.

In the foregoing, there is no special requirement for the linking region, so long as the linking region can connect the two connected parts of each of the R monomers and L monomers without affecting the functions of the two. The linking region can be a polypeptide. In the R monomer, the mr monomer, the reporter group YI, and the binding region 1 or a biomolecule containing the binding region 1 can be sequentially connected through the linking region or the chemical bond.

In the L monomer, the ml monomer, the reporter group BING, and the binding region 2 or a biomolecule containing the binding region 2 can be sequentially connected through the linking region or the chemical bond.

Each of the R monomers is connected to at least one biomolecule X.

In an embodiment of the present invention, the C-terminus of each of the R monomers is connected to the N-terminus of the biomolecule X through the linking region.

In the above reagent set 1 and reagent set 3, all the R monomers can be the same, all the L monomers can be the same and all the E monomers can be the same.

Both the mr monomer and the ml monomer can be yeast SmF. Yeast SmF protein is a core component of the ribonucleoprotein complex, and its crystal structure shows that it exists in the form of homo-tetradecamer. Therefore, using SmF as a carrier can achieve the multimerization of target proteins.

The mc monomer can be Bacillus subtilis protein Hfq. The Bacillus subtilis protein Hfq exists in the form of homohexamer. Therefore, using Hfq as a carrier can achieve the multimerization of target proteins.

The binding region 1 can be a region in SH3 as shown in positions 364-431 of SEQ ID NO: 1 that binds to PRMH as shown in positions 366-380 of SEQ ID NO: 5. The binding region 2 can be a region in PRMH as shown in positions 366-380 of SEQ ID NO: 5 that binds to SH3 as shown in positions 364-431 of SEQ ID NO: 1.

The linking region is (Gly-Gly-Ser)ₙ or a polypeptide containing (Gly-Gly-Ser)ₙ, and n is a natural number greater than or equal to 2.

n can specifically be 4 or 2.

The reporter group JIA can be a red fluorescent protein, such as mCherry.

The reporter group YI and the reporter group BING can be green fluorescent proteins, such as GFP.

In the above reagent set 1 and reagent set 3, both the mr monomer and the ml monomer can be yeast SmF as shown in positions 17-102 of SEQ ID NO: 1.

The mc monomer can be Hfq as shown in positions 17-94 of SEQ ID NO: 19.

The biomolecule containing the binding region 1 can be SH3 as shown in positions 364-431 of SEQ ID NO: 1.

The biomolecule containing the binding region 2 can be PRMH as shown in positions 366-380 of SEQ ID NO: 5.

In the above reagent set 1 and reagent set 3, the R monomer can be the following H1) or H2) or H3):

H1) a protein having the amino acid sequence as shown in positions 17-431 of SEQ ID NO: 1;
H2) a protein obtained by substitution and/or deletion and/or addition of one or more amino acid residues in the amino acid sequence as shown in positions 17-431 of SEQ ID NO: 1 in the Sequence Listing and having the same function;
H3) a fusion protein obtained by ligating tag(s) to the N-terminus or/and C-terminus of H1) or H2).

The L monomer is the following I1) or I2) or I3):

I1) a protein having the amino acid sequence as shown in positions 17-380 of SEQ ID NO: 5;
I2) a protein obtained by substitution and/or deletion and/or addition of one or more amino acid residues in the amino acid sequence as shown in positions 17-380 of SEQ ID NO: 5 in the Sequence Listing and having the same function;
I3) a fusion protein obtained by ligating tag(s) to the N-terminus or/and C-terminus of I1) or I2).

The E monomer is J1) or J2) or J3):

J1) a protein having the amino acid sequence as shown in positions 17-465 of SEQ ID NO: 19;
J2) a protein obtained by substitution and/or deletion and/or addition of one or more amino acid residues in the amino acid sequence as shown in positions 17-465 of SEQ ID NO: 19 in the Sequence Listing and having the same function;
J3) a fusion protein obtained by ligating tag(s) to the N-terminus or/and C-terminus of J1) or J2).

In order to facilitate the purification of the protein in H1) or I1) or J1), a tag as shown in Table 1 can be attached to the amino terminus or carboxyl terminus of H1) or I1) or J1).

TABLE 1
Tag sequences

| Tag | Residues | Sequence |
|---|---|---|
| Poly-Arg | 5-6 (usually 5) | RRRRR |
| Poly-His | 2-10 (usually 6) | HHHHHH |
| FLAG | 8 | DYKDDDDK |
| Strep-tag II | 8 | WSHPQFEK |
| c-myc | 10 | EQKLISEEDL |

For the protein in above H2) or I2) or J2), the substitution and/or deletion and/or addition of one or more amino acid residues is substitution and/or deletion and/or addition of no more than 10 amino acid residues.

The protein in above H2) or I2) or J2) can be artificially synthesized, or can be obtained by first synthesizing its encoding gene and then conducting biological expression.

The encoding gene of the protein in above H2) or I2) or J2) can be obtained by deleting the codons of one or more amino acid residues, and/or performing missense mutations of one or more base pairs, and/or attaching the encoding sequence(s) of the tag(s) shown in Table 1 to the 5′ end and/or the 3′ end in the DNA sequence encoding the R monomer or the DNA sequence encoding the L monomer or the DNA sequence encoding the E monomer in the present invention.

In order to solve the above technical problems, the present invention further provides a reagent set named reagent set 2 or reagent set 4 (i.e., the reagent set 2 in the Chinese Application No. 201711315673.6), and the reagent set 2 consists of the following X1) and X2) and the reagent set 4 consists of the following X1), X2), X3) and X4):

X1) a biological material related to the R monomer, which is any one of the following X11) to X14):
X11) a nucleic acid molecule encoding the R monomer:
X12) an expression cassette containing the nucleic acid molecule of X11);
X13) a recombinant vector containing the nucleic acid molecule of X11), or a recombinant vector containing the expression cassette of X12);
X14) a recombinant microorganism containing the nucleic acid molecule of X11), or a recombinant microorganism containing the expression cassette of X12), or a recombinant microorganism containing the recombinant vector of X13);
X2) a biological material related to the L monomer, which is any one of the following X21) to X24):
X21) a nucleic acid molecule encoding the L monomer;
X22) an expression cassette containing the nucleic acid molecule of X21);
X23) a recombinant vector containing the nucleic acid molecule of X21), or a recombinant vector containing the expression cassette of X22);
X24) a recombinant microorganism containing the nucleic acid molecule of X21), or a recombinant microorganism containing the expression cassette of X22), or a recombinant microorganism containing the recombinant vector of X23).
X3) a biological material related to the E monomer, which is any one of the following X31) to X34):
X31) a nucleic acid molecule encoding the E monomer;
X32) an expression cassette containing the nucleic acid molecule of X31);
X33) a recombinant vector containing the nucleic acid molecule of X31), or a recombinant vector containing the expression cassette of X32);
X34) a recombinant microorganism containing the nucleic acid molecule of X31), or a recombinant microorganism containing the expression cassette of X32), or a recombinant microorganism containing the recombinant vector of X33).
X4) a biological material related to the biomolecule Y_{D}, which is any one of the following X41) to X44):
X41) a nucleic acid molecule encoding the biomolecule Y_{D}:
X42) an expression cassette containing the nucleic acid molecule of X41);
X43) a recombinant vector containing the nucleic acid molecule of X41), or a recombinant vector containing the expression cassette of X42);
X44) a recombinant microorganism containing the nucleic acid molecule of X41), or a recombinant microorganism containing the expression cassette of X42), or a recombinant microorganism containing the recombinant vector of X43).

The reagent set 2 and reagent set 4 can be used to detect or assist in detecting whether there is an interaction between the biomolecule X and the biomolecule X_{L}, and can also be used to identify or assist in identifying a regulatory factor for the interaction between the biomolecule X and the biomolecule X_{L}.

In the above reagent set 2 and reagent set 4, the nucleic acid molecule of X11) can be the following x11) or x12) or x13):

x11) a cDNA molecule or a DNA molecule having the encoding sequence in positions 62-1306 of SEQ ID NO: 2 in the Sequence Listing;
x12) a cDNA molecule or a genomic DNA molecule having 75% or more identity with the nucleotide sequence defined by x11) and encoding the R monomer;
x13) a cDNA molecule or a genomic DNA molecule hybridizing to the nucleotide sequence defined by x11) under stringent conditions and encoding the R monomer.

The nucleic acid molecule of X21) can be the following x21) or x22) or x23):

x21) a cDNA molecule or a DNA molecule having the encoding sequence in positions 62-1153 of SEQ ID NO: 6 in the Sequence Listing;
x22) a cDNA molecule or a genomic DNA molecule having 75% or more identity with the nucleotide sequence defined by x21) and encoding the L monomer;
x23) a cDNA molecule or a genomic DNA molecule hybridizing to the nucleotide sequence defined by x21) under stringent conditions and encoding the L monomer.

The nucleic acid molecule of X31) can be the following x31) or x32) or x33):

x31) a cDNA molecule or a DNA molecule having the encoding sequence in positions 51-1400 of SEQ ID NO: 20 in the Sequence Listing;
x32) a cDNA molecule or a genomic DNA molecule having 75% or more identity with the nucleotide sequence defined by x31) and encoding the E monomer;
x33) a cDNA molecule or a genomic DNA molecule hybridizing to the nucleotide sequence defined by x31) under stringent conditions and encoding the E monomer.

Wherein, the nucleic acid molecule can be DNA, such as cDNA, genomic DNA, or recombinant DNA; the nucleic acid molecule can also be RNA, such as mRNA or hnRNA.

The term “identity” as used herein refers to sequence similarity to a natural nucleotide sequence. “Identity” includes a nucleotide sequence having 75% or higher, or 85% or higher, or 90% or higher, or 95% or higher identity with the nucleotide sequence encoding the R monomer or the L monomer of the present invention. Identity can be evaluated with the naked eye or computer software. Using computer software, the identity between two or more sequences can be expressed as a percentage (%), which can be used to evaluate the identity between related sequences.

The stringent conditions are: in a solution of 2×SSC, 0.1% SDS, hybridizing at 68° C. and washing the membrane twice, 5 min each time, and then in a solution of 0.5×SSC, 0.1% SDS, hybridizing at 68° C. and washing the membrane twice, 15 min each time; or, in a solution of 0.1×SSPE (or 0.1×SSC) 0.1% SDS, hybridizing at 65° C. and washing the membrane.

The above 75% or more identity can be 80%, 85%, 90%, or 95% or more identity.

The expression cassette containing the nucleic acid molecule encoding the R monomer of X12) (R monomer gene expression cassette) refers to a DNA molecule capable of expressing the R monomer in a host cell, and the DNA molecule can contain not only a promoter that initiates transcription of the R monomer gene, but also a terminator that terminates transcription of the R monomer gene. Further, the expression cassette can further contain an enhancer sequence.

The expression cassette containing the nucleic acid molecule encoding the L monomer of X22) (L monomer gene expression cassette) refers to a DNA molecule capable of expressing the L monomer in a host cell, and the DNA molecule can contain not only a promoter that initiates transcription of the L monomer gene, but also a terminator that terminates transcription of the L monomer gene. Further, the expression cassette can further contain an enhancer sequence.

The expression cassette containing the nucleic acid molecule encoding the E monomer of X32) (E monomer gene expression cassette) refers to a DNA molecule capable of expressing the E monomer in a host cell, and the DNA molecule can contain not only a promoter that initiates transcription of the E monomer gene, but also a terminator that terminates transcription of the E monomer gene. Further, the expression cassette can further contain an enhancer sequence.

The recombinant vector containing the R monomer gene expression cassette or the L monomer gene expression cassette or the E monomer gene expression cassette can be constructed using an existing vector. The vector can be a plasmid, a cosmid, a phage, or a viral vector. The plasmid can specifically be a pRSFDuet-1 vector.

The recombinant vector of X13) can specifically be pRSFDuet-1-SGS, and the pRSFDuet-1-SGS is a recombinant vector obtained by replacing the DNA fragment (containing the recognition sequences of NcoI and XhoI) between the recognition sequences of NcoI and XhoI of the pRSFDuet-1 vector with a DNA molecule as shown in positions 12-1360 of SEQ ID NO: 2 in the Sequence Listing. The pRSFDuet-1-SGS can express a fusion protein of the R monomer and His-tag as shown in SEQ ID NO: 1.

The recombinant vector of X23) can specifically be pRSFDuet-1-SGP, and the pRSFDuet-1-SGP is a recombinant vector obtained by replacing the DNA fragment (containing the recognition sequences of NcoI and XhoI) between the recognition sequences of NcoI and XhoI of the pRSFDuet-1 vector with a DNA molecule as shown in positions 12-1162 of SEQ ID NO: 6 in the Sequence Listing. The pRSFDuet-1-SGS can express a fusion protein of the L monomer and His-tag as shown in SEQ ID NO: 5.

The recombinant vector of X33) can specifically be pRSFDuet-1-Hfq-mCherry-PDZ, and the pRSFDuet-1-Hfq-mCherry-PDZ is a recombinant vector obtained by replacing the DNA fragment (containing the recognition sequences of NcoI and XhoI) between the recognition sequences of NcoI and XhoI of the pRSFDuet-1 vector with a DNA molecule as shown in SEQ ID NO: 20 in the Sequence Listing. The pRSFDuet-1-Hfq-mCherry-PDZ can express the fusion protein formed by Hfq, mCherry, PDZ and His-tag as shown in SEQ ID NO: 19.

The microorganism can be yeast, bacteria, algae or fungus. Wherein, the bacteria can be E. coli.

In the above reagent set 3 and reagent set 4, the modification can be a protein post-translational modification or a de-modification of protein post-translational modification. The protein post-translational modification can be methylation, acetylation, phosphorylation, ubiquitination or glycosylation modification. The de-modification of protein post-translational modification can be demethylation, deacetylation, dephosphorylation, deubiquitination or deglycosylation.

In order to solve the above technical problems, the present invention further provides a method for detecting whether there is an interaction between biomolecules and the biomolecules are two biomolecules named X and X_{L}, respectively, and the method comprises the following steps:

a solution to be tested is obtained by mixing solution A, solution B and solution C; the solution A is a solution containing the reagent A; the solution B is a solution containing the reagent B; the solution C is a solution containing the reagent C; the biomolecule R in the reagent A and the biomolecule L in the reagent B in the solution to be tested interact to produce phase transition droplets; according to whether there is a signal of the reporter group JIA in the phase transition droplets in the solution to be tested, the interaction between the biomolecule X and the biomolecule X_{L} is determined: if there is a signal of the reporter group JIA in the phase transition droplets in the solution to be tested, the biomolecule X and the biomolecule X_{L} have an interaction or are supposed to have an interaction; if there is no signal of the reporter group JIA in the phase transition droplets in the solution to be tested, the biomolecule X and the biomolecule X_{L} have no interaction or are supposed to have no interaction.

In the above method, the biomolecule X_{L} is a modified protein, and the biomolecule X is a protein, and the method comprises the following steps:

a solution to be tested is obtained by mixing solution A, solution B, solution E and solution D; the solution E is a solution containing the reagent E; the solution D is a solution containing the reagent D; the biomolecule R in the reagent A and the biomolecule L in the reagent B in the solution to be tested interact to produce phase transition droplets; according to whether there is a signal of the reporter group JIA in the phase transition droplets in the solution to be tested, the interaction between the biomolecule X and the biomolecule X_{L} is determined: if there is a signal of the reporter group JIA in the phase transition droplets in the solution to be tested, the biomolecule X and the biomolecule X_{L} have an interaction or are supposed to have an interaction; if there is no signal of the reporter group JIA in the phase transition droplets in the solution to be tested, the biomolecule X and the biomolecule X_{L} have no interaction or are supposed to have no interaction.

Wherein, whether there is a signal of the reporter group JIA in the phase transition droplets in the solution to be tested refers to whether the signal of the reporter group JIA in the solution to be tested is enriched in the phase transition droplets, so that the signal of the reporter group JIA in the phase transition droplets is higher than that in the non-phase transition droplets in the solution to be tested. Specifically, determining whether there is an interaction between the biomolecule X and the biomolecule X_{L} according to the signal of the reporter group JIA in the phase transition droplets in the solution to be tested can comprise the following steps: if the signal of the reporter group JIA in the solution to be tested is enriched in the phase transition droplets, the biomolecule X and the biomolecule X_{L} have an interaction or are supposed to have an interaction; if the signal of the reporter group JIA in the solution to be tested is not enriched in the phase transition droplets, the biomolecule X and the biomolecule X_{L} have no interaction or are supposed to have no interaction.

The solution A can consist of the reagent A and a solvent, the solution B can consist of the reagent B and a solvent, the solution C can consist of the reagent C and a solvent, the solution E can consist of the reagent E and a solvent, the solution D can consist of the reagent D and a solvent, and the solvent can dissolve the reagent A, the reagent B, the reagent C, the reagent E and the reagent D.

In one embodiment of the present invention, the solvent is KMEI buffer, and the KMEI buffer is composed of a solvent and solutes, wherein the solvent is water and the solutes and their concentrations are: 150 mM KCl, 1 mM MgCl₂, 1 mM EGTA, 10 mM imidazole, 1 mM DTT, pH=7.

In one embodiment of the present invention, the protein is expressed by fusion with the known multivalent protein SmF from yeast to realize the multimerization of the target protein, that is, the multivalentization of the reagent A and the reagent B. The R monomer is SGS (SGS is an abbreviation of the fusion protein SmF-GFP-SH3), and the L monomer is SGP (SGP is an abbreviation of the fusion protein SmF-GFP-PRMH). The interaction between SH3 and PRMH causes the interaction between the multivalent proteins SGS and SGP and then a phase transition occurs to produce phase transition droplets.

In one embodiment of the present invention, the protein is expressed by fusion with the known multivalent protein SmF from yeast or the protein Hfq from Bacillus subtilis to realize the multimerization of the protein, that is, the multivalentization of the reagent A, the reagent B and the reagent E.

In the above method, the modification can be a protein post-translational modification or a de-modification of protein post-translational modification. The protein post-translational modification can be methylation, acetylation, phosphorylation, ubiquitination or glycosylation modification. The de-modification of protein post-translational modification can be demethylation, deacetylation, dephosphorylation, deubiquitination or deglycosylation.

In order to solve the above technical problems, the present invention also provides a method for identifying a regulatory factor between biomolecules, wherein the biomolecules are two biomolecules named X and X_{L}, respectively, and there is an interaction between the biomolecule X and the biomolecule X_{L}, and the method comprises the following steps:

a solution to be tested is obtained by mixing solution A, solution B, solution C and a regulatory factor to be tested; a control solution is obtained by mixing the solution A, the solution B and the solution C; in the solution to be tested and the control solution, the biomolecule R in the solution A and the biomolecule L in the solution B interact to produce phase transition droplets; by comparing the signal intensity of the reporter group JIA in the phase transition droplets in the solution to be tested with that in the control solution, it is determined whether the regulatory factor to be tested has a regulatory effect on the interaction between the biomolecule X and the biomolecule X_{L}; if the signal of the reporter group JIA in the phase transition droplets in the solution to be tested is stronger than the signal of the reporter group JIA in the phase transition droplets in the control solution, the regulatory factor has or is supposed to have a promoting effect on the interaction between the biomolecule X and the biomolecule X_{L}; if the signal of the reporter group JIA in the phase transition droplets in the solution to be tested is weaker than the signal of the reporter group JIA in the phase transition droplets in the control solution, the regulatory factor has or is supposed to have an inhibitory effect on the interaction between the biomolecule X and the biomolecule X_{L}; if the signal intensity of the reporter group JIA in the phase transition droplets in the solution to be tested is the same as the signal intensity of the reporter group JIA in the phase transition droplets in the control solution, the regulatory factor has or is supposed to have no regulatory effect on the interaction between the biomolecule X and the biomolecule X_{L}.

In order to solve the above technical problems, the present invention also provides any one of the following uses of the reagent set 1, the reagent set 2, the reagent set 3 or the reagent set 4:

Z1) for detecting or assisting in detecting whether there is an interaction between biomolecules;
Z2) for screening regulatory factors for interactions between biomolecules;
Z3) for identifying or assisting in identifying regulatory factors for interactions between biomolecules;
Z4) for detecting the influence of substances on interactions between biomolecules:
Z5) for preparing products for detecting whether there is an interaction between biomolecules;
Z6) for preparing products for screening regulatory factors for interactions between biomolecules;
Z7) for preparing products for identifying regulatory factors for interactions between biomolecules:
Z8) for detecting or assisting in detecting whether there is an interaction between modified proteins and other biomolecules;
Z9) for screening regulatory factors for interactions between modified proteins and other biomolecules;
Z10) for identifying or assisting in identifying regulatory factors for interactions between modified proteins and other biomolecules;
Z11) for detecting the influence of substances on interactions between modified proteins and other biomolecules;
Z12) for detecting whether a protein has an enzyme activity involved in protein post-translational modification;
Z13) for preparing products for detecting whether there is an interaction between modified proteins and other biomolecules;
Z14) for preparing products for screening regulatory factors for interactions between modified proteins and other biomolecules;
Z15) for preparing products for identifying regulatory factors for interactions between modified proteins and other biomolecules;
Z16) for preparing products for detecting whether a protein has an enzyme activity involved in protein post-translational modification.

In the above use, the other biomolecule can be a protein.

In the above use, the modification can be a protein post-translational modification or a de-modification of protein post-translational modification. The protein post-translational modification can be methylation, acetylation, phosphorylation, ubiquitination or glycosylation modification. The de-modification of protein post-translational modification can be demethylation, deacetylation, dephosphorylation, deubiquitination or deglycosylation.

In the above use, the product can be a kit.

The method for screening regulatory factors for interactions between biomolecules can be high-throughput screening, and the method for identifying regulatory factors for interactions between biomolecules can also be high-throughput identification.

Another technical problem to be solved by the present invention is how to detect the interaction between biomolecules in a cell and screen regulatory factors that affect their interaction.

In order to solve the above technical problem, the present invention provides a method for detecting the interaction between biomolecules in a cell, the biomolecules to be tested are named X and X_{L}, the biomolecule X is a protein, a nucleic acid or a polysaccharide, and the biomolecule X_{L} is a protein, a nucleic acid or a polysaccharide, and the method comprises the following U1) and U2):

U1) connecting a biomolecule named R and the biomolecule X to obtain a recombinant molecule named R—X; the biomolecule R containing intrinsically disordered proteins/regions (IDPs/IDRs); connecting the biomolecule X_{L} and a reporter group named J to obtain a recombinant molecule named X_{L}-J;
U2) introducing the recombinant molecule R—X and the recombinant molecule X_{L}-J into a biological cell to obtain a recombinant cell, and detecting whether the signal of the reporter group J in the recombinant cell is accumulated in a second phase formed by the intrinsically disordered proteins/regions to determine whether there is an interaction between the biomolecule X and the biomolecule X_{L}; if the signal of the reporter group J is accumulated in the second phase, the biomolecule X and the biomolecule X_{L} have an interaction or are supposed to have an interaction; if the signal of the reporter group J is not accumulated in the second phase, the biomolecule X and the biomolecule X_{L} have no interaction or are supposed to have no interaction.

In U2), when the biomolecule X, the biomolecule X_{L} and the reporter group J are proteins, the step of introducing the recombinant molecule R—X and the recombinant molecule X_{L}-J into a biological cell can be introducing the encoding genes of the recombinant molecule R—X and the recombinant molecule X_{L}-J into the biological cell such that the obtained recombinant cell expresses the recombinant molecule R—X and the recombinant molecule X_{L}-J.

The present invention further provides a method for identifying regulatory factors for interactions between biomolecules in a cell, the biomolecules to be tested are named X and X_{L}, the biomolecule X is a protein, a nucleic acid or a polysaccharide, and the biomolecule X_{L} is a protein, a nucleic acid or a polysaccharide, there is an interaction between the biomolecule X and the biomolecule X_{L}, and the method comprises the following V1) and V2):

V1) connecting a biomolecule named R and the biomolecule X to obtain a recombinant molecule named R—X; the biomolecule R containing intrinsically disordered proteins/regions (IDPs/IDRs); connecting the biomolecule X_{L} and a reporter group named J to obtain a recombinant molecule named X_{L}-J;
V2) introducing the recombinant molecule R—X and the recombinant molecule X_{L}-J into a biological cell to obtain a recombinant cell; culturing the recombinant cell and adding a regulatory factor to be tested to the culture system of the recombinant cell to obtain a system to be tested; culturing the recombinant cell to obtain a control system; then detecting the signal intensity of the reporter group J in the recombinant cell in a second phase formed by the intrinsically disordered proteins/regions in the system to be tested and the control system to determine whether the regulatory factor to be tested has a regulatory effect on the interaction between the biomolecule X and the biomolecule X_{L}; if the signal of the reporter group J in the second phase in the system to be tested is stronger than the signal of the reporter group J in the second phase in the control system, the regulatory factor to be tested has or is supposed to have a promoting effect on the interaction between the biomolecule X and the biomolecule X_{L}; if the signal intensity of the reporter group J in the second phase in the system to be tested is the same as the signal intensity of the reporter group J in the second phase in the control system, the regulatory factor to be tested has or is supposed to have no regulatory effect on the interaction between the biomolecule X and the biomolecule X_{L}; if the signal of the reporter group J in the phase transition droplets in the solution to be tested is weaker than the signal of the reporter group J in the second phase in the control system, the regulatory factor to be tested has or is supposed to have an inhibitory effect on the interaction between the biomolecule X and the biomolecule X_{L}.

In the above method, the biomolecule R can further contain a reporter group named K, and the reporter group K is different from the reporter group J.

In the above method, both the reporter group J and the reporter group K can be fluorescent reporter groups.

In the above method, the fluorescent reporter group can be a fluorescent protein.

Further, the reporter group J can be specifically a red fluorescent protein mCherry, and the reporter group K can be a green fluorescent protein GFP.

In the above method, the intrinsically disordered proteins/regions can be the following H1) or H2) or H3):

H1) a protein having the amino acid sequence as shown in positions 258-772 of SEQ ID NO: 24;
H2) a protein obtained by substitution and/or deletion and/or addition of one or more amino acid residues in the amino acid sequence as shown in positions 258-772 of SEQ ID NO: 24 in the Sequence Listing and having the same function;
H3) a fusion protein obtained by ligating tag(s) to the N-terminus or/and C-terminus of H1) or H2).

In order to facilitate the purification of the protein in H1), a tag as shown in Table 1 can be attached to the amino terminus or carboxyl terminus of H1).

For the protein in above H2), the substitution and/or deletion and/or addition of one or more amino acid residues is substitution and/or deletion and/or addition of no more than 10 amino acid residues.

The protein in above H2) can be artificially synthesized, or can be obtained by first synthesizing its encoding gene and then conducting biological expression.

The encoding gene of the protein in above H2) can be obtained by deleting the codons of one or more amino acid residues, and/or performing missense mutations of one or more base pairs, and/or attaching the encoding sequence(s) of the tag(s) shown in Table 1 to the 5′ end and/or the 3′ end in the DNA sequence encoding the intrinsically disordered proteins/regions.

In the above method, the reporter group K in the biomolecule R and the intrinsically disordered proteins/regions can be connected through a linking region or a chemical bond.

In the above method, the biomolecule X_{L} and the reporter group J in the recombinant molecule X_{L}-J can be connected through a linking region or a chemical bond.

The biomolecule R and the biomolecule X in the recombinant molecule R—X can be connected through a linking region or a chemical bond.

In the above method, the linking region can be (Gly-Gly-Ser)ₙ or a polypeptide containing (Gly-Gly-Ser)ₙ, and n is a natural number greater than or equal to 2.

n can specifically be 4 or 2.

In the above method, the biomolecule R can be the following I1), I2), I3) or I4):

I1) a protein having the amino acid sequence as shown in positions 1-772 of SEQ ID NO: 24;
I2) a protein having the amino acid sequence as shown in positions 1-784 of SEQ ID NO: 24;
I3) a protein obtained by substitution and/or deletion and/or addition of one or more amino acid residues in the amino acid sequence as shown in positions 1-772 or 1-784 of SEQ ID NO: 24 in the Sequence Listing and having the same function:
I4) a fusion protein obtained by ligating tag(s) to the N-terminus or/and C-terminus of I1), I2) or I3).

In order to facilitate the purification of the protein in I1), a tag as shown in Table 1 can be attached to the amino terminus or carboxyl terminus of I1).

For the protein in above I2), the substitution and/or deletion and/or addition of one or more amino acid residues is substitution and/or deletion and/or addition of no more than 10 amino acid residues.

The protein in above I2) can be artificially synthesized, or can be obtained by first synthesizing its encoding gene and then conducting biological expression.

The encoding gene of the protein in above I2) can be obtained by deleting the codons of one or more amino acid residues, and/or performing missense mutations of one or more base pairs, and/or attaching the encoding sequence(s) of the tag(s) shown in Table 1 to the 5′ end and/or the 3′ end in the DNA sequence encoding the biomolecule R.

In the above method, the biological cell can be an animal cell, a plant cell, or a microbial cell. In one embodiment of the present invention, the animal cell is a HEK293 cell.

In one embodiment of the present invention, the biomolecule X is p53, and the biomolecule X_{L} is MDM2.

The present invention also provides the biomolecule R.

The present invention also provides a biological material related to the biomolecule R, and the biological material is any one of the following M1) to M4):

M1) a nucleic acid molecule encoding the biomolecule R;
M2) an expression cassette containing the nucleic acid molecule of M1);
M3) a recombinant vector containing the nucleic acid molecule of M1), or a recombinant vector containing the expression cassette of M2);
M4) a recombinant microorganism containing the nucleic acid molecule of M1), or a recombinant microorganism containing the expression cassette of M2), or a recombinant microorganism containing the recombinant vector of M3).

In the above biological material, the nucleic acid molecule of M1) can be any one of the following m1)-m8):

m1) a cDNA molecule or a DNA molecule having the encoding sequence as shown in positions 780-2324 of SEQ ID NO: 25 in the Sequence Listing;
m2) a cDNA molecule or a DNA molecule having the encoding sequence as shown in positions 738-2324 of SEQ ID NO: 25 in the Sequence Listing;
m3) a cDNA molecule or a DNA molecule having the encoding sequence as shown in positions 9-2324 of SEQ ID NO: 25 in the Sequence Listing;
m4) a cDNA molecule or a DNA molecule having the encoding sequence as shown in positions 780-2360 of SEQ ID NO: 25 in the Sequence Listing;
m5) a cDNA molecule or a DNA molecule having the encoding sequence as shown in positions 738-2360 of SEQ ID NO: 25 in the Sequence Listing;
m6) a cDNA molecule or a DNA molecule having the encoding sequence as shown in positions 9-2360 of SEQ ID NO: 25 in the Sequence Listing;
m7) a cDNA molecule or a DNA molecule having 75% or more identity with the nucleotide sequence defined by m1) or m2) or m3) or m4) or m5) or m6) and encoding the biomolecule R;
m8) a cDNA molecule or a DNA molecule hybridizing to the nucleotide sequence defined by m1) or m2) or m3) or m4) or m5) or m6) under stringent conditions and encoding the biomolecule R.

Wherein, the nucleic acid molecule can be DNA, such as cDNA, genomic DNA, or recombinant DNA; the nucleic acid molecule can also be RNA, such as mRNA or hnRNA.

The term “identity” as used herein refers to sequence similarity to a natural nucleotide sequence. “Identity” includes a nucleotide sequence having 75% or higher, or 85% or higher, or 90% or higher, or 95% or higher identity with the nucleotide sequence encoding the biomolecule R of the present invention. Identity can be evaluated with the naked eye or computer software. Using computer software, the identity between two or more sequences can be expressed as a percentage (%), which can be used to evaluate the identity between related sequences.

The stringent conditions are: in a solution of 2×SSC, 0.1% SDS, hybridizing at 68° C. and washing the membrane twice, 5 min each time, and then in a solution of 0.5×SSC, 0.1% SDS, hybridizing at 68° C. and washing the membrane twice, 15 min each time; or, in a solution of 0.1×SSPE (or 0.1×SSC), 0.1% SDS, hybridizing at 65° C. and washing the membrane.

The above 75% or more identity can be 80%, 85%, 90%, or 95% or more identity.

The expression cassette containing the nucleic acid molecule encoding the biomolecule R of M2) (R gene expression cassette) refers to a DNA molecule capable of expressing the biomolecule R in a host cell, and the DNA molecule can contain not only a promoter that initiates transcription of the R gene, but also a terminator that terminates transcription of the R gene. Further, the expression cassette can further contain an enhancer sequence.

The recombinant vector containing the R gene expression cassette can be constructed using an existing vector. The vector can be a plasmid, a cosmid, a phage, or a viral vector. The plasmid can specifically be a pcDNA3.1 vector.

The recombinant vector of X13) can specifically be pcDNA3.1-GFP-NUPN, and the pcDNA3.1-GFP-NUPN is a recombinant vector obtained by replacing the DNA fragment (containing the recognition sequences of NotI and XbaI) between the recognition sequences of NotI and XbaI of the pcDNA3.1 vector with a DNA molecule as shown in SEQ ID NO: 25 in the Sequence Listing. The pcDNA3.1-GFP-NUPN can express a fusion protein GFP-NUPN of GFP and NUPN as shown in SEQ ID NO: 24.

The microorganism can be yeast, bacteria, algae or fungus.

The present invention also provides any one of the following uses of the biomolecule R or the biological material:

X1) for detecting the interaction between biomolecules in a cell:
X2) for preparing products for detecting the interaction between biomolecules in a cell;
X3) for identifying regulatory factors for interactions between biomolecules in a cell;
X4) for preparing products for identifying regulatory factors for interactions between biomolecules in a cell;
X5) for screening regulatory factors for interactions between biomolecules in a cell;
X6) for preparing products for screening regulatory factors for interactions between biomolecules in a cell;
X7) for detecting the influence of substances on interactions between biomolecules in a cell.

In the above use, the cell can be an animal cell, a plant cell, or a microbial cell. In one embodiment of the present invention, the animal cell is HEK293 cell.

In the above use, the product can be a kit.

The method for screening regulatory factors for interactions between biomolecules can be high-throughput screening, and the method for identifying regulatory factors for interactions between biomolecules can also be high-throughput identification.

### DESCRIPTION OF THE DRAWINGS

FIG. 1 shows the detection of the interaction between P53 and MDM2. Panel A is the morphologic observation of the phase transition droplets of system 3, and the right image is an enlarged image of the selected area in the left image; panel B is the confocal high-content microscopic imaging analysis of systems 1-11. Scale bar=50 μm.

FIG. 2 shows the effect of inhibitor on the interaction between P53 and MDM2. Panel A is the detection result of the fluorescence signal, and panel B is the quantitative analysis of the red fluorescence signal in the phase transition droplets. Scale bar=20 μm.

FIG. 3 shows the effect of rapamycin on the interaction between FKBP and FRB. Panel A is the detection result of the fluorescence signal, and panel B is the quantitative analysis of the red fluorescence signal in the phase transition droplets. Scale bar=100 μm.

FIG. 4 shows the analysis of the experimental results of the detection systems 1-8 for the interaction between H3K9me3 and CD. Panel A is the morphologic observation of the phase transition droplets of system 3, and the right image is an enlarged image of the selected area in the left image. Panel B is the confocal high-content microscopic imaging analysis of systems 1-8. Scale bar=100 μm.

FIG. 5 shows the analysis of the experimental results of the detection systems 9-20 for the interaction between H3K9me3 and CD. Panel A is the confocal high-content microscopic imaging analysis of systems 9-20. Scale bar=100 μm. Panel B is a quantitative analysis of the mCherry fluorescence intensity in the phase transition droplets in panel A. H3K9me3 and H3K9 represent H3K9me3-KKETPV and H3K9-KKETPV, respectively; 0, 0.2, 0.4, 0.6, 0.8, and 1.0 represent the proportion of H3K9me3-KKETPV in the mixture of H3K9me3-KKETPV and H3K9-KKETPV.

FIG. 6 shows the detection of the interaction between P53 and MDM2. Panel A is the morphologic observation the second phase generated by system 1, and the right image is an enlarged image of the selected area in the left image; panel B is the laser-scanning confocal microscopy imaging analysis of systems 1-8. Scale bar=20 μm.

FIG. 7 is a laser-scanning confocal microscopy imaging analysis of the effect of the inhibitor on the interaction between P53 and MDM2. Scale bar=20 μm.

### DETAILED DESCRIPTION OF THE INVENTION

The following further describes the present invention in detail with reference to specific embodiments. The examples are given only for illustrating the present invention, but not for limiting the scope of the present invention. Unless otherwise specified, the experimental methods in the following examples are conventional methods. The materials, reagents and instruments, etc. used in the following examples are available commercially, unless otherwise specified. For the quantitative experiments in the following examples, the experiments are all repeated three times and the results are averaged. In the following examples, unless otherwise specified, the nucleotide at the first position of each nucleotide sequence in the Sequence Listing refers to the nucleotide at the 5′ end of the corresponding DNA, and the nucleotide at the last position refers to the nucleotide at the 3′ end of the corresponding DNA.

In the following examples, the pcDNA3.1 vector (Yoo et al., A new strategy for assessing selenoprotein function: siRNA knockdown/knock-in targeting the 3′-UTR, RNA (2007), 13: 921-929.) is available to the public from the applicant. This biological material is only used for repeating the relevant experiments of the present invention, and cannot be used for other purposes.

In the following examples, the HEK293 cell (SHIN et al., Overexpression of PGC-1α enhances cell proliferation and tumorigenesis of HEK293 cells through the upregulation of Sp1 and Acyl-CoA binding protein, INTERNATIONAL JOURNAL OF ONCOLOGY 46: 1328-1342, 2015) is available to the public from the applicant. This biological material is only used for repeating the relevant experiments of the present invention, and cannot be used for other purposes.

Examples 1 and 2 provide a reagent set for detecting the interaction between a biomolecule X and a biomolecule X_{L}, and the reagent set consists of reagent A, reagent B and reagent C:

the reagent A is formed by connecting a biomolecule R and a biomolecule X, the biomolecule R is a polymer formed by R monomers, all the R monomers are the same, each monomer contains a mr monomer, a fluorescent reporter group YI, a binding region 1 or a biomolecule containing binding region 1, and the parts of each monomer are connected by a linking region or a chemical bond, wherein two or more mr monomers can form a polymer;
the reagent B contains a biomolecule L, the biomolecule L is a polymer formed by L monomers, all the L monomers are the same, each monomer contains a m1 monomer, a fluorescent reporter group BING, a binding region 2 or a biomolecule containing binding domain 2, and the parts of each monomer are connected by a linking region or a chemical bond, wherein two or more m1 monomers can form a polymer;
the reagent C is formed by connecting a fluorescent reporter group JIA and a biomolecule X_{L};
the biomolecule R and the biomolecule L are the same or different and there is an interaction between the two. The interaction between the biomolecule R and the biomolecule L is carried out by the binding region 1 of the biomolecule R and the binding region 2 of the biomolecule L, the number of the binding region 1 in the biomolecule R and the number of the binding region 2 in the biomolecule L are both greater than or equal to 2, and a phase transition occurs when the biomolecule R interacts with the biomolecule L;
the biomolecule X and the biomolecule X_{L} are proteins, nucleic acids or polysaccharides; the biomolecule R and the biomolecule L are proteins, nucleic acids or polysaccharides.

The following uses biomolecules R and L (both being proteins) as examples to illustrate the use of the reagent set of the present invention for detecting protein-protein interactions. Specifically, the mr monomer and the m1 monomer are both SmF, the fluorescent reporter group YI and the fluorescent reporter group BING are both GFP, the biomolecule containing binding region 1 is SH3, the biomolecule containing binding region 2 is PRMH, the fusion protein of SmF. GFP and SH3 is denoted as SGS (i.e., R monomer), and the fusion protein of SmF, GFP and PRMH is denoted as SGP (i.e., L monomer), the fluorescent reporter group JIA is mCherry.

### Example 1. Detection of Interaction Between P53 and MDM2 and Effect of Inhibitor on it

In this example, the biomolecule X is P53, and the biomolecule X_{L} is MDM2.

I. Preparation of Recombinant Vectors

1. Recombinant Vector Expressing Fusion Protein of SGS and P53

The DNA fragment (containing the recognition sequences of NcoI and XhoI) between the recognition sequences of NcoI and XhoI of the pRSFDuet-1 vector (purchased from Novagen, Merck) was replaced with the DNA molecule as shown in positions 12-1360 of SEQ ID NO: 2 in the Sequence Listing to obtain a recombinant vector pRSFDuet-1-SGS, and the pRSFDuet-1-SGS was capable of expressing the protein as shown in SEQ ID NO: 1 (SGS fused with His-tag, i.e., R monomer, denoted as His-SGS).

Wherein, the DNA molecule as shown in positions 14-1354 of SEQ ID NO: 2 encodes His-SGS as shown in SEQ ID NO: 1, and the DNA sequences as shown in positions 1344-1349 and 1355-1360 of SEQ ID NO: 2 are the recognition sequences of NcoI and XhoI, respectively; the sequence as shown in positions 3-8 of SEQ ID NO: 1 is the amino acid sequence of His-tag, the sequence as shown in positions 17-102 of SEQ ID NO: 1 is the amino acid sequence of SmF, the sequence as shown in positions 109-349 of SEQ ID NO: 1 is the amino acid sequence of GFP, the sequence as shown in positions 364-431 of SEQ ID NO: 1 is the amino acid sequence of SH3, the sequences as shown in positions 103-108, 350-363 and 432-444 of SEQ ID NO: 1 are the amino acid sequences of the linking region. His-SGS can form tetradecamer through the action of SmF.

The DNA fragment (containing the recognition sequences of NcoI and XhoI) between the recognition sequences of NcoI and XhoI of the pRSFDuet-1-SGS was replaced with the DNA molecule as shown in positions 5-66 of SEQ ID NO: 4 in the Sequence Listing to obtain a recombinant vector pRSFDuet-1-SGS-P53, and the pRSFDuet-1-SGS-P53 was capable of expressing a fusion protein (denoted as SGS-P53) of His-SGS as shown in SEQ ID NO: 1 and P53 as shown in SEQ ID NO: 3 in the Sequence Listing.

Wherein, the DNA molecule as shown in positions 13-57 of SEQ ID NO: 4 encodes P53 as shown in SEQ ID NO: 3. SGS-P53 can form tetradecamer through the action of SmF.

2. Recombinant Vector Expressing SGP

The DNA fragment (containing the recognition sequences of NcoI and XhoI) between the recognition sequences of NcoI and XhoI of the pRSFDuet-1 vector was replaced with the DNA molecule as shown in positions 12-1162 of SEQ ID NO: 6 in the Sequence Listing to obtain a recombinant vector pRSFDuet-1-SGP, and the pRSFDuet-1-SGP was capable of expressing the protein as shown in SEQ ID NO: 5 (SGP fused with His-tag, i.e. L monomer, denoted as His-SGP).

Wherein, the DNA molecule as shown in positions 14-1156 of SEQ ID NO: 6 encodes His-SGP as shown in SEQ ID NO: 5; the sequence as shown in positions 3-8 of SEQ ID NO: 5 is the amino acid sequence of His-tag, the sequence as shown in positions 17-102 of SEQ ID NO: 5 is the amino acid sequence of SmF, the sequence as shown in positions 109-349 of SEQ ID NO: 5 is the amino acid sequence of GFP, the sequence as shown in positions 366-380 of SEQ ID NO: 5 is the amino acid sequence of PRMH, the sequences as shown in positions 103-108 and 350-365 of SEQ ID NO: 5 are the amino acid sequences of the linking region. His-SGP can form tetradecamer through the action of SmF.

3. Recombinant Vector Expressing Fusion Protein of MDM2 and mCherry (Reagent C)

The DNA fragment (containing the recognition sequences of NcoI and XhoI) between the recognition sequences of NcoI and XhoI of the pRSFDuet-1 vector was replaced with the DNA molecule as shown in positions 11-831 of SEQ ID NO: 8 in the Sequence Listing to obtain a recombinant vector pRSFDuet-1-mCherry, and the pRSFDuet-1-mCherry was capable of expressing the protein as shown in SEQ ID NO: 7 (mCherry fused with His-tag, denoted as His-mCherry).

Wherein, the sequence as shown in positions 13-825 of SEQ ID NO: 8 encodes His-mCherry as shown in SEQ ID NO: 7, the sequences in positions 815-820 and 826-831 of SEQ ID NO: 8 are the recognition sequences of NcoI and XhoI, respectively; the sequence as shown in positions 3-8 of SEQ ID NO: 7 is the amino acid sequence of His-tag, the sequence as shown in positions 17-254 of SEQ ID NO: 7 is the amino acid sequence of mCherry, and the sequence as shown in positions 255-270 of SEQ ID NO: 7 is the amino acid sequence of the linking region.

The DNA fragment (containing the recognition sequences of NcoI and XhoI) between the recognition sequences of NcoI and XhoI of the pRSFDuet-1-mCherry was replaced with the DNA molecule as shown in positions 5-324 of SEQ ID NO: 10 to obtain a recombinant vector pRSFDuet-1-mCherry-MDM2, and the pRSFDuet-1-mCherry-MDM2 was capable of expressing a fusion protein of mCherry and MDM2 shown in SEQ ID NO: 9 (i.e., reagent C, which was denoted as mCherry-MDM2 and contained a His-tag).

Wherein, the sequence as shown in positions 7-315 of SEQ ID NO: 10 encodes MDM2 as shown in SEQ ID NO: 9.

The DNA fragment (containing the recognition sequences of NcoI and XhoI) between the recognition sequences of NcoI and XhoI of the pRSFDuet-1-mCherry was replaced with the DNA molecule as shown in positions 1-35 of SEQ ID NO:16 to obtain a recombinant vector pRSFDuet-1-mCherry-N, and the pRSFDuet-1-mCherry-N was capable of expressing a fusion protein of mCherry and the protein as shown in SEQ ID NO: 15 (this fusion protein was denoted as mCherry-N and contains a His-tag).

II. Expression and Purification of Fusion Proteins

The pRSFDuet-1-SGS, pRSFDuet-1-SGS-P53, pRSFDuet-1-SGP, pRSFDuet-1-mCherry, pRSFDuet-1-mCherry-MDM2 and pRSFDuet-1-mCherry-N vectors obtained in step I were respectively introduced into E. coli competent cells BL21 (DE3) (TIANGEN BIOTECH (BEIJING) CO., LTD) to obtain recombinant strains BL21-pRSFDuet-1-SGS, BL21-pRSFDuet-1-SGS-P53, BL21-pRSFDuet-1-SGP, BL21-pRSFDuet-1-mCherry, BL21-pRSFDuet-1-mCherry-MDM2 and BL21-pRSFDuet-1-mCherry-N.

The fusion proteins containing His-tag expressed by the recombinant strains BL21-pRSFDuet-1-SGS, BL21-pRSFDuet-1-SGS-P53, BL21-pRSFDuet-1-SGP, BL21-pRSFDuet-1-mCherry, BL21-pRSFDuet-1-mCherry-MDM2 and BL21-pRSFDuet-1-mCherry-N were purified, according to the following steps:

(1) Bacteria culture and protein induction expression: each of the above recombinant strains was inoculated into 1 L LB medium, incubated at 37° C., 200 rpm until the OD600 reached about 0.8-1 (about 8-9 hr); the bacterial culture was incubated at 18° C. for 1 hr for cooling and IPTG was added to a final concentration of 0.5 mM to induce protein expression, then the culture was incubated overnight (about 16 hr), and then the bacterial culture was obtained.
(2) Suspension and lysis of bacteria: the bacterial culture obtained in step (1) was centrifuged, and the supernatant was discarded; the bacterial pellet was resuspended in 40 mL binding buffer (40 mM Tris-Cl, 500 mM NaCl, pH 8.0 or 7.4) and sonicated; the lysate was ultracentrifuged at 20000 rpm for 1 hr and the supernatant (containing the target fusion protein) was collected.
(3) Ni column purification: a Ni column was prepared in advance and equilibrated with the binding buffer; the supernatant obtained in step (2) was loaded onto the Ni column; when the liquid was almost dry, the wash buffer was added to wash 2-3 column volumes, then the elution buffer was added to elute the target fusion protein, and the effluent was collected.
wash buffer: 40 mM Tris-HCl, 500 mM NaCl, 40 mM imidazole, pH was the same as the binding buffer.
elution buffer: 40 mM Tris-HCl, 500 mM NaCl, 500 mM imidazole, pH was the same as the binding buffer.
(4) Ion exchange purification: based on the isoelectric point of the protein, a suitable ion exchange column was selected; the effluent obtained in step (3) was diluted with 40 mM Tris-Cl buffer to reduce the ion concentration to obtain a protein diluent; the ion exchange column was installed in the ATKA protein purification system (GE company) and the protein diluent was loaded; the protein bound to the column was eluted by gradually increasing the salt ion concentration and the target fusion protein was collected; the eluent used for the elution was composed of solution A and solution B, and the ratio between the two was adjusted according to specific conditions: buffer A: 40 mM Tris-Cl, pH was the same as the binding buffer; buffer B: 40 mM Tris-Cl, 2M NaCl, pH was the same as the binding buffer.
(5) Protein purification by gel filtration: the target fusion protein obtained in step (4) was concentrated by ultrafiltration, and then was separated and purified by a preset gel filtration program to obtain a further purified target fusion protein.

The KMEI buffer used for column equilibration and elution was composed of a solvent and solutes. The solvent was water and the solutes and their concentrations were: 150 mM KC, 1 mM MgCl₂, 1 mM EGTA, 10 mM imidazole, 1 mM DTT, pH=7.

(6) Detection and storage of purified proteins: His-SGS expressed by the BL21-pRSFDuet-1-SGS, SGS-P53 expressed by the BL21-pRSFDuet-1-SGS-P53, His-SGP expressed by the BL21-pRSFDuet-1-SGP, His-mCherry expressed by the BL21-pRSFDuet-1-mCherry, mCherry-MDM2 expressed by the BL21-pRSFDuet-1-mCherry-MDM2 and mCherry-N expressed by the BL21-pRSFDuet-1-mCherry-N, which were obtained by the purification of the above step, were detected by SDS-PAGE. After the fusion proteins were confirmed to have the expected sizes, the proteins were concentrated and frozen at −80° C. until use.

III. Detection of Interaction Between P53 and MDM2

The solutions of His-SGS, SGS-P53, His-SGP, His-mCherry, mCherry-N and mCherry-MDM2 obtained in step 11 (all solvents being KMEI buffer) were respectively loaded into a 384-microwell plate according to the following systems, one system per well. The concentrations of His-SGS, His-SGP, His-mCherry, SGS-P53, mCherry-MDM2 and mCherry-N in the corresponding systems were all 0.5 μM:
system 1: the solution of His-SGS;
system 2: the solution of His-SGP;
system 3: the solution of His-SGS and His-SGP;
system 4: the solution of His-mCherry;
system 5: the solution of His-SGS, His-mCherry and His-SGP;
system 6: the solution of SGS-P53;
system 7: the solution of SGS-P53 and His-SGP;
system 8: the solution of SGS-P53, His-mCherry and His-SGP;
system 9: the solution of SGS-P53, mCherry-MDM2 and His-SGP;
system 10: the solution of His-SGS, mCherry-MDM2 and His-SGP;
system 11: the solution of SGS-P53, mCherry-N and His-SGP.

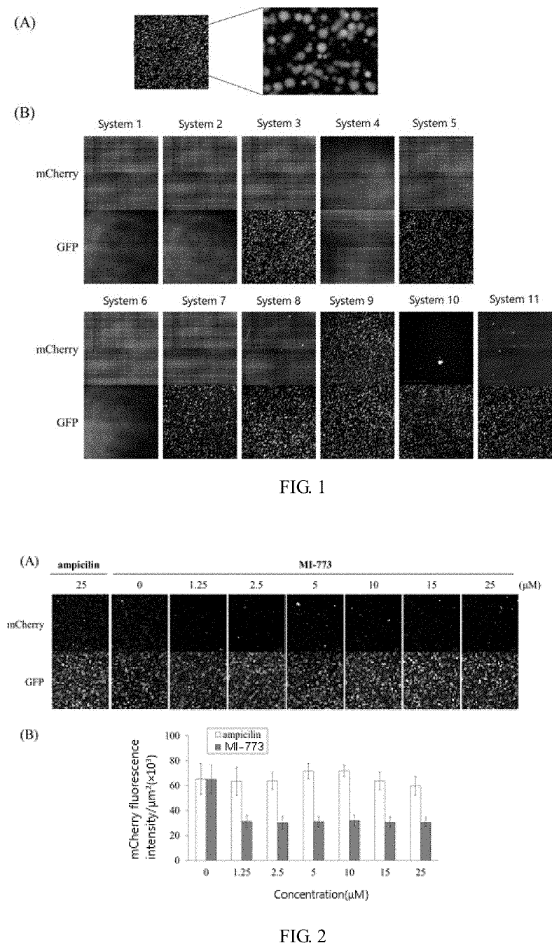

Each of the above systems was subjected to static incubation at 4° C. until the phase transition droplets in the systems in which phase transitions occurred completely settled to the bottom of the well plate, and the images were collected using a confocal high-content imaging microscope. The result (panel B in FIG. 1) showed that the solutions in the system 1 and the system 2 had no change, and no fluorescence signal accumulation area was found; the solution in the system 3 generated phase transition droplets, and the green fluorescent signal (the fluorescent signal from GFP) was accumulated in the phase transition droplets, and the signal intensity in the droplets was much higher than the signal intensity in the solution (panel A in FIG. 1); the solution in the system 4 had no change, and no fluorescence signal accumulation area was found; the solution in the system 5 generated phase transition droplets, and the green fluorescence signal was accumulated in the phase transition droplets, and the signal intensity in the droplets was much higher than the signal intensity in the solution, and at the same time, no red fluorescence signal accumulation was found in the droplets; the solution in the system 6 had no change, and no fluorescence signal accumulation area was found; the solution in the system 7 generated phase transition droplets, the green fluorescence signal was accumulated in the phase transition droplets, and the signal intensity in the droplets was much higher than the signal intensity in the solution; the solution in the system 8 generated phase transition droplets, the green fluorescence signal was accumulated in the phase transition droplets, and the signal intensity in the droplets was much higher than the signal intensity in the solution, and at the same time no red fluorescence signal accumulation was found in the droplets; the solution in the system 9 generated phase transition droplets, both the green fluorescence signal and the red fluorescence signal (the fluorescence signal from mCherry) were accumulated in the phase transition droplets, and the signal intensity in the droplets was much higher than the signal intensity in the solution; the solutions in systems 10 and 11 generated phase transition droplets, the green fluorescent signal was accumulated in the phase transition droplets and the signal intensity in the droplets was much higher than the signal intensity in the solution, and at the same time no red fluorescence signal accumulation was found in the droplets. The above results show that SGS can bind with SGP to generate phase transition droplets marked with fluorescence emitted by GFP, when the phase transition droplets contain the protein P53 which can interact with MDM2, P53 can recruit mCherry-MDM2 to the phase transition droplets by interacting with MDM2, and then the red fluorescence signal is accumulated in the phase transition droplets. In the absence of system components or the addition of proteins with no interaction with P53, the accumulation of the red fluorescence signal could not be detected. It shows that the interaction between P53 and MDM2 can be detected using SGS-P53, mCherry-MDM2 and His-SGP.

IV. Validation of MI-773 Inhibiting Interaction Between P53 and MDM2

In the following system, the compound MI-773, which is known to have an inhibitory effect on the interaction between P53 and MDM2, was used and its effect on the interaction between P53 and MDM2 was verified by utilizing the method of the present invention and ampicillin was used as a control:

the solutions of SGS-P53, mCherry-MDM2, His-SGP and ampicillin were mixed, and then 20 μL of the resulting mixture was transferred to a 384-microwell plate to obtain an ampicillin control system and the concentrations of SGS-P53, mCherry-MDM2 and His-SGP were all 0.5 μM in the ampicillin control system, and the concentration of ampicillin was 25 μM;

According to the above system, the solutions of SGS-P53, mCherry-MDM2, His-SGP and MI-773 were mixed, and then 20 μL of the resulting mixture was transferred into a 384-microwell plate. Different concentrations of MI-773 were set to obtain experimental systems with different concentrations of MI-773. The concentration of MI-773 in each experimental system was 0 (i.e., KMEI buffer), 1.25, 2.5, 5, 10, 15, and 25 μM, respectively, one concentration per well, and in each experimental system, the concentrations of SGS-P53, mCherry-MDM2 and His-SGP were all 0.5 μM.

Each of the above systems was subjected to static incubation at 4° C. until the phase transition droplets in the systems in which phase transitions occurred completely settled to the bottom of the well plate, and the image of each system was collected using a confocal high-content imaging microscope and the intensity of the red fluorescence signal in the phase transition droplets in each well plate was counted and analyzed. The result (FIG. 2) showed that after adding the inhibitor MI-773, the red fluorescence signal in the phase transition droplets was significantly weaker than that of the system containing ampicillin but no MI-773, indicating that ampicillin does not affect the interaction between P53 and MDM2 while MI-773 can significantly inhibit the interaction between P53 and MDM2, which is consistent with the known MI-773's inhibitory effect on the interaction between P53 and MDM2. It indicates that SGS-P53, mCherry-MDM2 and His-SGP can be used to detect the influence of regulatory factors on the interaction between P53 and MDM2.

### Example 2. Verification of Rapamycin Promoting Interaction Between FKBP and FRB

In this example, the biomolecule X is FKBP, and the biomolecule X_{L} is FRB.

I. Preparation of Recombinant Vectors

The DNA fragment (containing the recognition sequences of NcoI and XhoI) between the recognition sequences of NcoI and XhoI of the pRSFDuet-1-SGS of Example 1 was replaced with the DNA molecule as shown in SEQ ID NO: 12 in the Sequence Listing to obtain a recombinant vector pRSFDuet-1-SGS-FKBP, and the pRSFDuet-1-SGS-FKBP was capable of expressing a fusion protein (denoted as SGS-FKBP) of SGS as shown in SEQ ID NO: 1 and FKBP as shown in SEQ ID NO: 11 in the Sequence Listing.

Wherein, the sequence as shown in positions 3-332 of SEQ ID NO:12 encodes FKBP as shown in SEQ ID NO: 11.

The DNA fragment between the recognition sequences of NcoI and XhoI of the pRSFDuet-1-mCherry of Example 1 was replaced with the DNA molecule as shown in SEQ ID NO: 14 to obtain a recombinant vector pRSFDuet-1-mCherry-FRB, and the pRSFDuet-1-mCherry-FRB was capable of expressing a fusion protein of mCherry and FRB as shown in SEQ ID NO: 13 (i.e., reagent C, which was denoted as mCherry-FRB and contained a His-tag).

Wherein, the sequence as shown in positions 3-293 of SEQ ID NO: 14 encodes FRB as shown in SEQ ID NO: 13.

II. Expression and Purification of Fusion Proteins

According to the method of step I in Example 1, pRSFDuet-1-SGS-FKBP and pRSFDuet-1-mCherry-FRB were introduced into E. coli competent cells BL21 (DE3), respectively, and then expression and purification of SGS-FKBP and mCherry-FRB were performed to obtain a solution of SGS-FKBP and a solution of mCherry-FRB both in KMEI buffer.

III. Effect of Rapamycin on Interaction Between FKBP and FRB

The method of the present invention was used to verify the effect of rapamycin on the interaction between FKBP and FRB according to the following systems, and ampicillin was used as a control:

the solutions of SGS-FKBP, mCherry-FRB, His-SGP and ampicillin were mixed, and then 20 μL of the resulting mixture was transferred to a 384-microwell plate to obtain an ampicillin control system and the concentrations of SGS-FKBP, mCherry-FRB, His-SGP and ampicillin in the ampicillin control system were all 1 LM;

According to the above system, the solutions of SGS-FKBP, mCherry-FRB, His-SGP and rapamycin were mixed, and then 20 μL of the resulting mixture was transferred into a 384-microwell plate. Different concentrations of rapamycin were set to obtain experimental systems with different concentrations of rapamycin. The concentration of rapamycin in each experimental system was 0 (i.e., KMEI buffer), 0.2, 0.4, 0.6, 0.8, and 1.0 μM, one concentration per well, and in each experimental system, the concentrations of SGS-FKBP, mCherry-FRB and His-SGP were all 1 μM.

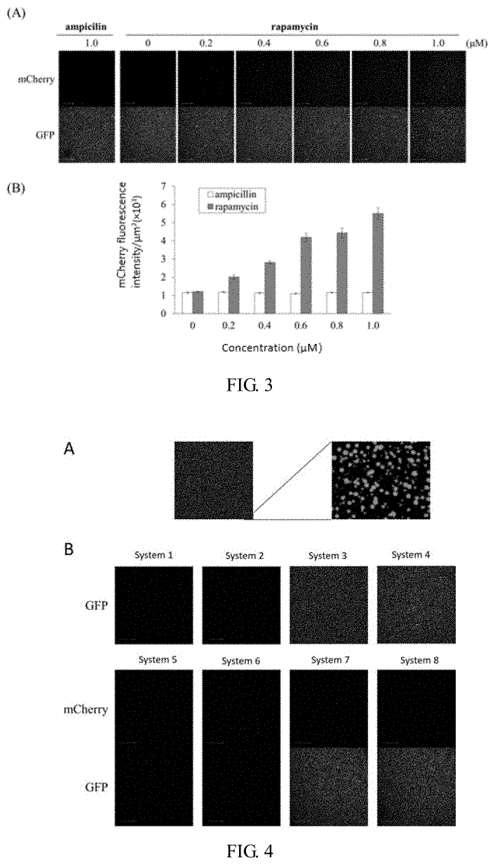

Each of the above systems was subjected to static incubation at 4° C. until the phase transition droplets in the systems in which phase transitions occurred completely settled to the bottom of the well plate, and the image of each system was collected using a confocal high-content imaging microscope and the intensity of the red fluorescence signal in the phase transition droplets in each well plate was counted and analyzed. The results (FIG. 3) showed that in each rapamycin-added system, the red fluorescence signal in the phase transition droplets was significantly stronger than that in the system containing ampicillin but no rapamycin and the intensity of the red fluorescence signal in the phase transition droplets increased with the increase of rapamycin concentration. It indicated that ampicillin does not affect the interaction between FRB and FKBP, rapamycin can promote the interaction between FRB and FKBP, and the promotion effect has dose effect, which is consistent with the known rapamycin's promoting effect on the interaction between FKBP and FRB. It shows that the effects of regulatory factors on the interaction between FRB and FKBP can be detected using SGS-FKBP, mCherry-FRB and His-SGP.

Example 3 provides a reagent set for detecting interactions between a biomolecule X and a biomolecule X_{L}, and the reagent set consists of four reagents named A, B. E and D, respectively;

the reagent A is formed by connecting a biomolecule named R and a protein named X, the biomolecule R is a polymer formed by R monomers, all the R monomers are the same, each monomer contains a mr monomer, a fluorescent reporter group named YI, a binding region 1 or a biomolecule containing binding region 1, and the parts of each monomer are connected by a linking region or a chemical bond, wherein two or more mr monomers can form a polymer;
the reagent B contains a biomolecule named L, the biomolecule L is a polymer formed by L monomers, all the L monomers are the same, each monomer contains a monomer ml, a fluorescent reporter group named BING, a binding region 2 or a biomolecule containing binding domain 2, and the parts of each monomer are connected by a linking region or a chemical bond, wherein two or more m1 monomers can form a polymer:
the biomolecule R and the biomolecule L are the same or different and there is an interaction between the two; the interaction between the biomolecule R and the biomolecule L is carried out by the binding region 1 of the biomolecule R and the binding region 2 of the biomolecule L, the number of the binding region 1 in the biomolecule R and the number of the binding region 2 in the biomolecule L are both greater than or equal to 2, and a phase transition occurs when the biomolecule R interacts with the biomolecule L;
the reagent E is a polymer formed by E monomers, and the E monomer is formed by connecting a monomer named mc, a fluorescent reporter group named JIA and a biomolecule named Y_{C}, wherein two or more mc monomers can form a polymer;
the reagent D is formed by connecting a post-translationally modified protein named X_{L} and a biomolecule named Y_{D}:
there is an interaction between the biomolecule Y_{C} and the biomolecule Y_{D}.

The following uses the biomolecules R and L (both being proteins) as examples to illustrate the use of the reagent set of the present invention for detecting the interaction between a methylated protein and its ligand. Specifically, the mr monomer and m1 the monomer are both SmF, the fluorescent reporter group YI and the fluorescent reporter group BING are both GFP, the biomolecule containing binding region 1 is SH3, the biomolecule containing binding region 2 is PRMH, the fusion protein of SmF, GFP and SH3 is denoted as SGS (i.e., R monomer), the fusion protein of SmF, GFP and PRMH is denoted as SGP (i.e., L monomer), the mc monomer is Hfq, the fluorescent reporter group JIA is mCherry, the biomolecule Yc is PDZ with the sequence as shown in positions 362-465 of SEQ ID NO: 19; Hfq, mCherry and PDZ are fused to obtain the E monomer; Y_{D} is KKETPV (as shown in positions 22-29 of SEQ ID NO: 23).

### Example 3. Verification of Interaction Between H3K9Me3 and CD

In this example, the biomolecule X_{L} is H3K9me3, the biomolecule X is CD, and there is an interaction between the H3K9me3 and CD.

I. Preparation of Recombinant Vectors

1. Recombinant Vector Expressing Fusion Protein of SGS and CD

The preparation of the pRSFDuet-1-SGS was the same as step I in Example 1.

The DNA fragment (containing the recognition sequences of NcoI and XhoI) between the recognition sequences of NcoI and XhoI of the pRSFDuet-1-SGS was replaced with the DNA molecule as shown in positions 1-212 of SEQ ID NO: 18 in the Sequence Listing to obtain a recombinant vector pRSFDuet-1-SGS-CD, and the pRSFDuet-1-SGS-CD was capable of expressing a fusion protein (denoted as SGS-CD) of His-SGS as shown in SEQ ID NO: 1 and CD as shown in SEQ ID NO: 17 in the Sequence Listing.

Wherein, the sequence as shown in positions 9-203 of SEQ ID NO: 18 encodes CD as shown in SEQ ID NO: 17. SGS-CD can form tetradecamer through the action of SmF.

2. Recombinant Vector Expressing SGP

The preparation of the pRSFDuet-1-SGP was the same as step I in Example 1.

3. Recombinant Vector Expressing E Monomer (Hfq-mCherry-PDZ)

The DNA fragment (containing the recognition sequences of NcoI and XhoI) between the recognition sequences of NcoI and XhoI of the pRSFDuet-1 vector was replaced with the DNA molecule as shown in SEQ ID NO: 20 in the Sequence Listing to obtain a recombinant vector pRSFDuet-1-Hfq-mCherry-PDZ, and the pRSFDuet-1-Hfq-mCherry-PDZ was capable of expressing the protein as shown in SEQ ID NO: 19 (a fusion protein formed by Hfq, mCherry, PDZ, and His-tag, denoted as Hfq-mCherry-PDZ).

Wherein, the DNA molecule as shown in positions 3-1397 of SEQ ID NO: 20 encodes Hfq-mCherry-PDZ as shown in SEQ ID NO: 19, the sequence as shown in positions 3-8 of SEQ ID NO: 19 is the amino acid sequence of His-tag, the sequence as shown in positions 17-94 of SEQ ID NO: 19 is the amino acid sequence of Hfq, the sequence as shown in positions 101-340 of SEQ ID NO: 19 is the amino acid sequence of mCherry, the sequence as shown in positions 362-465 of SEQ ID NO: 19 is the amino acid sequence of PDZ, and the sequences as shown in positions 95-100 and 341-361 of SEQ ID NO: 19 are the amino acid sequences of the linker region.

Hfq-mCherry-PDZ can form hexamer through the action of Hfq.

Preparation of a recombinant vector expressing a fusion protein containing no Hfq: the DNA fragment (containing the recognition sequences of NcoI and XhoI) between the recognition sequences of NcoI and XhoI of the pRSFDuet-1 vector was replaced with the DNA molecule as shown in positions 11-1143 of SEQ ID NO: 22 in the Sequence Listing to obtain a recombinant vector pRSFDuet-1-mCherry-PDZ, and the pRSFDuet-1-mCherry-PDZ was capable of expressing the protein as shown in SEQ ID NO: 21 (the fusion protein formed by mCherry, PDZ and His-tag, denoted as mCherry-PDZ, as a control below).

Wherein, the DNA molecule as shown in positions 13-1134 of SEQ ID NO: 22 encodes mCherry-PDZ as shown in SEQ ID NO: 21; the sequence as shown in positions 3-8 of SEQ ID NO: 21 is the amino acid sequence of His-tag, the sequence as shown in positions 17-256 of SEQ ID NO: 21 is the amino acid sequence of mCherry, the sequence as shown in positions 271-374 of SEQ ID NO: 21 is the amino acid sequence of PDZ, and the sequence as shown in positions 257-270 of SEQ ID NO: 21 is the amino acid sequence of the linker region.

4. Preparation of Reagent D

The reagent D formed by connecting a protein X_{L} and a biomolecule Y_{D} was chemically synthesized and the reagent D was a methylated protein obtained by trimethylating lysine at position 4 of H3K9-KKETPV as shown in SEQ ID NO: 23 in the Sequence Listing and was denoted as H3K9me3-KKETPV. The sequence of H3K9me3-KKETPV is as follows: ARTK (Me) 3QTARGGSGGSGGSWGGSKKETPVAV.

II. Expression and Purification of Fusion Proteins

The pRSFDuet-1-SGS, pRSFDuet-1-SGS-CD, pRSFDuet-1-SGP, pRSFDuet-1-Hfq-mCherry-PDZ and pRSFDuet-1-mCherry-PDZ vectors obtained in step I were respectively introduced into E. coli competent cells BL21 (DE3) (TIANGEN BIOTECH (BEIJING) CO., LTD) to obtain recombinant strains BL21-pRSFDuet-1-SGS, BL21-pRSFDuet-1-SGS-CD, BL21-pRSFDuet-1-SGP, BL21-pRSFDuet-1-Hfq-mCherry-PDZ and BL21-pRSFDuet-1-mCherry-PDZ.

The fusion proteins containing His-tag expressed by the recombinant strains BL21-pRSFDuet-1-SGS, BL21-pRSFDuet-1-SGS-CD, BL21-pRSFDuet-1-SGP, BL21-pRSFDuet-1-Hfq-mCherry-PDZ and BL21-pRSFDuet-1-mCherry-PDZ were purified, according to the following method:

(1) Bacteria culture and protein induction expression: Each of the above recombinant strains was inoculated into 1 L LB medium, incubated at 37° C., 200 rpm to reach an OD600 of about 0.8-1 (about 8-9 hr); the bacterial culture was incubated at 18° C. for 1 hr for cooling and IPTG was added to a final concentration of 0.5 mM to induce protein expression, then the culture was incubated overnight (about 16 hr), and then the bacterial culture was obtained.
(2) Suspension and lysis of bacteria: the bacterial culture obtained in step (1) was centrifuged, and the supernatant was discarded; the bacterial pellet was resuspended in 40 mL binding buffer (40 mM Tris-Cl, 500 mM NaCl, pH 8.0 or 7.4) and sonicated; the lysate was ultracentrifuged at 20000 rpm for 1 hr and the supernatant (containing the target fusion protein) was collected.
(3) Ni column purification: a Ni column was prepared in advance and equilibrated with the binding buffer. The supernatant obtained in step (2) was loaded onto the Ni column; when the liquid was almost dry, the wash buffer was added to wash 2-3 column volumes, then the elution buffer was added to elute the target fusion protein, and the effluent was collected.
wash buffer: 40 mM Tris-HCl, 500 mM NaCl, 40 mM imidazole, pH was the same as the binding buffer.
elution buffer: 40 mM Tris-HC, 500 mM NaCl, 500 mM imidazole, pH was the same as the binding buffer.
(4) Ion exchange purification: based on the isoelectric point of the protein, a suitable ion exchange column was selected; the effluent obtained in step (3) was diluted with 40 mM Tris-Cl buffer to reduce the ion concentration to obtain a protein diluent; the ion exchange column was installed in the ATKA protein purification system (GE company) and the protein diluent was loaded; the protein bound to the column was eluted by gradually increasing the salt ion concentration and the target fusion protein was collected; the eluent used for the elution was composed of buffer A and buffer B, and the ratio between the two was adjusted according to specific conditions: buffer A: 40 mM Tris-Cl, pH was the same as the binding buffer; buffer B: 40 mM Tris-Cl, 2M NaCl, pH was the same as the binding buffer.
(5) Protein purification by gel filtration: the target fusion protein obtained in step (4) was concentrated by ultrafiltration, and then was separated and purified by a preset gel filtration program to obtain a further purified target fusion protein.

The KMEI buffer used for column equilibration and elution was composed of a solvent and solutes. The solvent was water and the solutes and their concentrations were: 150 mM KCl, 1 mM MgCl₂, 1 mM EGTA, 10 mM imidazole, 1 mM DTT, pH=7.

(6) Detection and storage of purified proteins: His-SGS expressed by BL21-pRSFDuet-1-SGS, SGS-CD expressed by the BL21-pRSFDuet-1-SGS-CD, His-SGP expressed by the pRSFDuet-1-SGP, Hfq-mCherry-PDZ expressed by the BL21-pRSFDuet-1-Hfq-mCherry-PDZ, and mCherry-PDZ expressed by the BL21-pRSFDuet-1-mCherry-PDZ, which were obtained by the purification of the above step, were detected by SDS-PAGE. After the fusion proteins were confirmed to have the expected sizes, the proteins were concentrated and frozen at −80° C. until use.

III. Detection of Interaction Between H3K9Me3 and CD

The solutions of His-SGS, SGS-CD, His-SGP, Hfq-mCherry-PDZ, mCherry-PDZ (as a control), H3K9me3-KKETPV and H3K9-KKETPV (as a control) obtained in step 11 (all solvents being KMEI buffer) were respectively loaded into a 384-microwell plate according to the systems as shown in Table 2, one system per well. The concentrations of His-SGS, SGS-CD, His-SGP, Hfq-mCherry-PDZ, mCherry-PDZ and the mixture of H3K9me3-KKETPV and H3K9-KKETPV in the corresponding systems were all 1 μM:

TABLE 2
Systems for detecting the interaction between H3K9me3 and CD

| | SGS- | His- | His- | mCherry- | Hfq-mCherry- | H3K9me3- | H3K9- |
|---|---|---|---|---|---|---|---|
| System | CD | SGS | SGP | PDZ | PDZ | KKETPV | KKETPV |
| 1 | − | + | − | − | − | − | − |
| 2 | − | − | + | − | − | − | − |
| 3 | − | + | + | − | − | − | − |
| 4 | + | − | + | − | − | − | − |
| 5 | − | − | − | + | − | − | − |
| 6 | − | − | − | − | + | − | − |
| 7 | + | − | + | − | − | + | − |
| 8 | + | − | + | − | + | − | − |

| | | | | | | |
|---|---|---|---|---|---|---|
| 9 | + | − | + | + | − | * |
| 10 | + | − | + | + | − | * |
| 11 | + | − | + | + | − | * |
| 12 | + | − | + | + | − | * |
| 13 | + | − | + | + | − | * |
| 14 | + | − | + | + | − | * |
| 15 | + | − | + | − | + | * |
| 16 | + | − | + | − | + | * |
| 17 | + | − | + | − | + | * |
| 18 | + | − | + | − | + | * |
| 19 | + | − | + | − | + | * |
| 20 | + | − | + | − | + | * |

In Table 2, “+” represents that the substance is contained; “−” represents that the substance is not contained; “*” represents a mixture of H3K9me3-KKETPV and H3K9-KKETPV. In the systems 9-14, the molar percentages of H3K9me3-KKETPV in the mixture of H3K9me3-KKETPV and H3K9-KKETPV were 0, 0.2, 0.4, 0.6, 0.8 and 1.0, respectively, and in the systems 15-20, the molar percentages of H3K9me3-KKETPV in the mixture of H3K9me3-KKETPV and H3K9-KKETPV were 0, 0.2, 0.4, 0.6, 0.8 and 1.0, respectively.

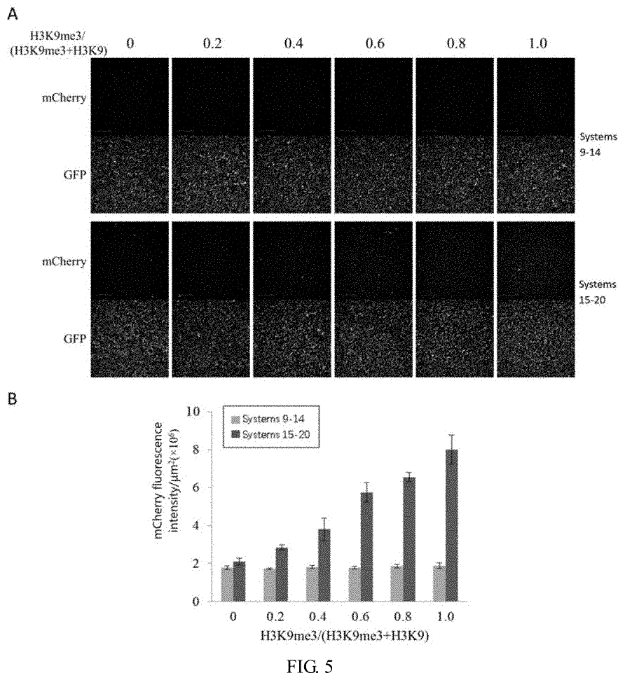

Each of the above systems was subjected to a static incubation at 4° C. until the phase transition droplets in the systems in which phase transitions occurred completely settled to the bottom of the well plate, and the images were collected using a confocal high-content imaging microscope. The result (panel Bin FIG. 4) showed that the solutions in the system 1 and the system 2 had no change, and no fluorescence signal accumulation area was found; the solutions in the system 3 and the system 4 both generated phase transition droplets, and the green fluorescent signal (the fluorescent signal from GFP) was accumulated in the phase transition droplets, and the signal intensity in the droplets was much higher than the signal intensity in the solution (panel A in FIG. 4); the solutions in the system 5 and the system 6 had no change, and no fluorescence signal accumulation area was found; the solutions in the system 7 and the system 8 both generated phase transition droplets, and the green fluorescence signal was accumulated in the phase transition droplets, and the signal intensity in the droplets was much higher than the signal intensity in the solution, and at the same time, no red fluorescence signal accumulation was found in the droplets; the systems 9-14 generated phase transition droplets, the green fluorescence signal was accumulated in the phase transition droplets, and the signal intensity in the droplets was much higher than the signal intensity in the solution and at the same time, no significant red fluorescence signal accumulation was found in the droplets (FIG. 5); the systems 15-20 generated phase transition droplets, the green fluorescence signal was accumulated in the phase transition droplets, and the signal intensity in the droplets was much higher than the signal intensity in the solution; at the same time, it was found that when the mixture was completely composed of H3K9-KKETPV (that is, when the molar percentage of H3K9me3-KKETPV in the mixture was 0), there was no significant red fluorescence (the fluorescence signal from mCherry) in the phase transition droplets; however, with the proportion of H3K9me3 in the mixture of H3K9me3 and H3K9 increased, the intensity of the red fluorescence signal in the phase transition droplets also increased; until the mixture was completely composed of H3K9me3-KKETPV (that is, when the molar percentage of H3K9me3-KKETPV in the mixture was 1.0), the red fluorescence signal intensity in the phase transition droplets reached the highest (FIG. 5)

It shows that SGS can bind with SGP to generate phase transition droplets, and the phase transition droplets can be marked with a fluorescent signal from GFP. In the system where mCherry-PDZ exists and Hfq-mCherry-PDZ does not exist, no matter whether the system contains H3K9me3-KKETPV or H3K9-KKETPV, the red fluorescence signal cannot be accumulated in the phase transition droplets in this system. In the system where mCherry-PDZ does not exist but Hfq-mCherry-PDZ exists, the content of H3K9me3 is positively correlated with the degree of accumulation of the red fluorescence signal in the phase transition droplets. It shows that CD in SGS-CD can recruit H3K9me3-KKETPV into the phase transition droplets through the interaction between CD and H3K9me3, and KKETPV in H3K9me3-KKETPV can further recruit Hfq-mCherry-PDZ that emits a red fluorescent signal into the phase transition droplets, so an accumulation of red fluorescence signal was detected in the phase transition droplets; if Hfq-mCherry-PDZ is replaced with mCherry-PDZ, the accumulation of red fluorescence signal in the phase transition droplets is not obvious, indicating that hexavalent Hfq-mCherry-PDZ is more suitable for detecting the interaction between CD and H3K9me3 than monovalent mCherry-PDZ.

In summary, the phase transition-based reagent set and multivalent recruitment system of this example have the characteristics of amplifying the interaction signal and improving the detection sensitivity of the interaction between H3K9me3 and CD, thereby providing a simple and convenient method for the detection of the weak interaction between post-translationally modified proteins and their ligands.

### Example 4. Intracellular Detection of Interaction Between P53 and MDM2 and Effect of Inhibitor on it

In this example, the biomolecule X is P53, and the biomolecule X_{L} is MDM2.

I. Preparation of Recombinant Vectors

1. Recombinant Vector Expressing Fusion Protein of GFP and NUPN (N-Terminus of NUP98)

The DNA fragment (containing the recognition sequences of NotI and XbaI) between the recognition sequences of NotI and XbaI of the pcDNA3.1 vector was replaced with the DNA molecule shown in SEQ ID NO: 25 in the Sequence Listing to obtain a recombinant vector pcDNA3.1-GFP-NUPN, and pcDNA3.1-GFP-NUPN was capable of expressing the protein shown in SEQ ID NO: 24 (GFP fused with NUPN, denoted as GFP-NUPN).

Wherein, the DNA molecule as shown in positions 9-2363 of SEQ ID NO: 25 encodes GFP-NUPN shown in SEQ ID NO: 24, the sequence as shown in positions 1-241 of SEQ ID NO: 24 is the amino acid sequence of GFP, the sequence as shown in positions 244-255 of SEQ ID NO: 24 is the amino acid sequence of a (GGS)₄ linker peptide, the sequence as shown in positions 258-772 of SEQ ID NO: 24 is the amino acid sequence of NUPN, and the sequence as shown in positions 773-784 of SEQ ID NO: 24 is the amino acid sequence of a (GGS)₄ linker peptide.

2. Recombinant Vector Expressing Fusion Protein of GFP-NUPN and P53

The DNA fragment (containing the recognition sequences of NotI and XbaI) between the recognition sequences NotI and XbaI of the pcDNA3.1 vector was replaced with the DNA molecule shown in SEQ ID NO: 27 in the Sequence Listing to obtain a recombinant vector pcDNA3.1-GFP-NUPN-p53, and pcDNA3.1-GFP-NUPN-p53 was capable of expressing the protein as shown in SEQ ID NO: 26 (GFP fused with NUPN and p53, denoted as GFP-NUPN-p53).

Wherein, the DNA molecule as shown in positions 9-2408 of SEQ ID NO: 27 encodes GFP-NUPN-p53 as shown in SEQ ID NO: 26, the sequence as shown in positions 1-241 of SEQ ID NO: 26 is the amino acid sequence of GFP, the sequence as shown in positions 244-255 of SEQ ID NO: 26 is the amino acid sequence of a (GGS)₄ linker peptide, the sequence as shown in positions 258-772 of SEQ ID NO: 26 is the amino acid sequence of NUPN, the sequence as shown in positions 773-784 of SEQ ID NO: 26 is the amino acid sequence of a (GGS)₄ linker peptide, and the sequence as shown in positions 785-799 of SEQ ID NO: 26 is the amino acid sequence of p53.

3. Recombinant Vector Expressing mCherry

The DNA fragment (containing the recognition sequences of Nod and XbaI) between the recognition sequences of NotI and XbaI of the pcDNA3.1 vector was replaced with the DNA molecule as shown in positions 1-785 of SEQ ID NO: 29 in the Sequence Listing to obtain a recombinant vector pcDNA3.1-mCherry, and pcDNA3.1-mCherry was capable of expressing the protein as shown in SEQ ID NO: 28 (mCherry fused with a (GGS)₄ linker peptide, denoted as mCherry-GGS).

Wherein, the DNA molecule as shown in positions 9-785 of SEQ ID NO: 29 encodes mCherry-GGS as shown in SEQ ID NO: 28, the sequence as shown in positions 1-238 of SEQ ID NO: 28 is the amino acid sequence of mCherry, and the sequence as shown in positions 241-252 of SEQ ID NO: 28 is the amino acid sequence of a (GGS)₄ linker peptide.

4. Recombinant Vector Expressing Fusion Protein of mCherry and MDM2

The DNA fragment (containing the recognition sequences of XhoI and XbaI) between the recognition sequences of XhoI and XbaI of the pcDNA3.1-mCherry vector was replaced with the DNA molecule as shown in SEQ ID NO: 30 in the Sequence Listing to obtain a recombinant vector pcDNA3.1-mCherry-MDM2, and the pcDNA3.1-mCherry-MDM2 was capable of expressing the fusion protein (denoted as mCherry-MDM2) of mCherry-GGS as shown in SEQ ID NO: 28 and MDM2 as shown in SEQ ID NO: 9.

II. Transfection of HEK293 Cells

Each recombinant vector obtained in step I was transfected (or co-transfected) into HEK293 cells (adherent culture) according to the following combinations, and the transfection reagent Hifectin I (Beijing Dinoao Biotechnology Co., Ltd.) was used, and the operation was performed according to the instruction of the reagent. The combinations and the amount of the transfection vector(s) per 105 HEK293 cells were as follows:

combination 1: pcDNA3.1-GFP-NUPN, the transfection dosage of the vector was 1 μg;
combination 2: pcDNA3.1-mCherry, the transfection dosage of the vector was 1 μg;
combination 3: pcDNA3.1-GFP-NUPN+pcDNA3.1-mCherry, the transfection dosages of these two vectors were 0.5 μg and 0.5 μg, respectively;
combination 4: pcDNA3.1-GFP-NUPN-P53, the transfection dosage of the vector was 1 μg;
combination 5: pcDNA3.1-GFP-NUPN-P53+pcDNA3.1-mCherry, the transfection dosages of these two vectors were 0.5 μg and 0.5 μg, respectively;
combination 6: pcDNA3.1-mCherry-MDM2, the transfection dosage of the vector was 1 μg;
combination 7: pcDNA3.1-GFP-NUPN+pcDNA3.1-mCherry-MDM2, the transfection dosages of these two vectors were 0.5 μg and 0.5 μg, respectively;
combination 8: pcDNA3.1-GFP-NUPN-P53+pcDNA3.1-mCherry-MDM2, the transfection dosages of these two vectors were 0.5 μg and 0.5 μg, respectively.

III. Intracellular Detection of Interaction Between P53 and MDM2

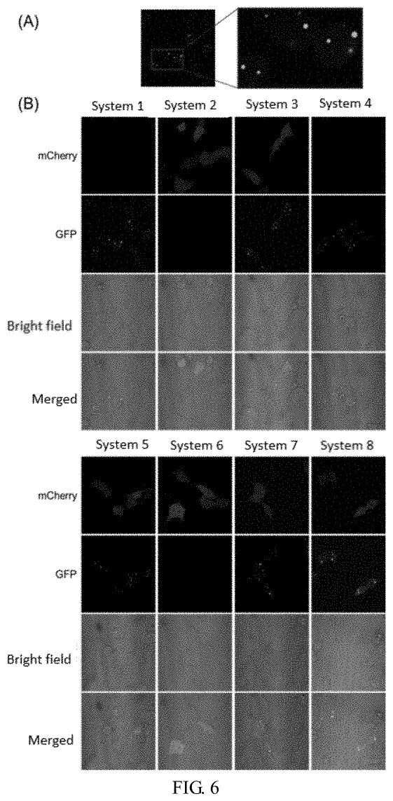

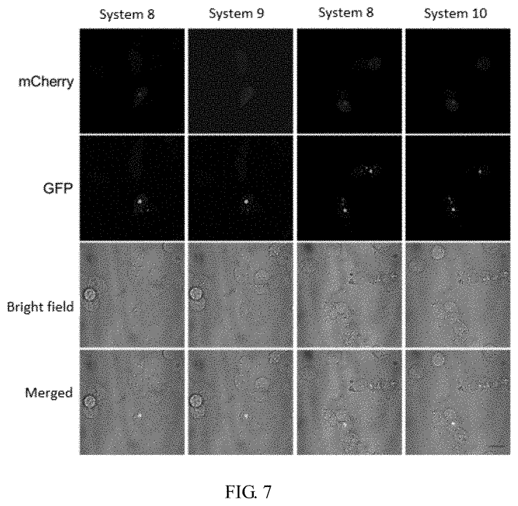

The transfected cell lines obtained in step II were cultured for 24 hours, and then images were collected using a laser-scanning confocal microscope. The results (FIG. 6) showed that in the systems 1 and 4 which were respectively transfected with combinations 1 and 4, phase transitions occurred in the cells, the green fluorescence signal (fluorescence signal from GFP) was accumulated in the second phase produced by the phase transition, and its signal intensity was much higher than the signal intensity of the non-phase transition part in the cell; in the systems 2 and 6 which were respectively transfected with combinations 2 and 6, the red fluorescence signal (fluorescence signal from mCherry) was uniformly distributed in the cells, and there was no accumulation; in the systems 3, 5 and 7 which were respectively transfected with combinations 3, 5 and 7, the phase transitions occurred in the cells, and the green fluorescence signal was accumulated in the second phase produced by the phase transition, and its signal intensity was much higher than that of the non-phase transition part in the cell, but the red fluorescence signal was not accumulated; in the system 8 which was transfected with combination 8, a phase transition occurred in the cells, both the green fluorescence signal and the red fluorescence signal were accumulated in the second phase produced by the phase transition, and their signal intensity was much higher than that of the non-phase transition part in the cell.

The above results show that NUPN can mediate the occurrence of intracellular phase transitions, the second phase produced by the phase transition can be marked by the fluorescence of GFP connected to NUPN; when NUPN, GFP and P53 are connected together, if MDM2 which can interact with P53 is contained in cells, P53 can recruit MDM2 connected with mCherry into the second phase through the interaction with MDM2, so that the red fluorescent signal is accumulated in the second phase. However, in the absence of P53 and/or MDM2, no accumulation of red fluorescence signal was detected. It shows that GFP-NUPN-P53 and mCherry-MDM2 can be used to co-transfect HEK293 cells to detect the intracellular interaction between P53 and MDM2.

IV. Validation of MI-773 Inhibiting Interaction Between P53 and MDM2

The compound MI-773, which is known to have an inhibitory effect on the interaction between P53 and MDM2, was used to treat the system 8 in step II, and its inhibitory effect on the interaction between P53 and MDM2 in the cells was tested, and the unrelated compound GDC0152 was used as a control. MI-773 (system 9) or GDC0152 (system 10) was added to the cell line of system 8 to a final concentration of 5 μM, respectively, and the same field of vision before and after treatment was acquired using a laser-scanning confocal microscope. The results (FIG. 7) showed that MI-773 treatment had no significant effect on the phase transition in the cells, the accumulation of the green fluorescent signal was still detected in the second phase, while the accumulation of the red fluorescent signal in the second phase disappeared and became uniformly distributed. However, both the phase transition state of the cells before and after GDC0152 treatment and the accumulation of the two fluorescent signals in the second phase did not change significantly. It shows that GDC0152 does not affect the interaction between P53 and MDM2, and M1-773 can significantly inhibit the interaction between P53 and MDM2, verifying MI-773's inhibitory effect on the interaction between P53 and MDM2. The above results indicate that the effects of regulatory factors on the interaction between P53 and MDM2 can be identified by co-transfection of HEK293 cells with GFP-NUPN-P53 and mCherry-MDM2.

### INDUSTRIAL APPLICATIONS

The present invention first prepares a reagent set that can be used to detect the interaction between biomolecules based on the phase transition, the biomolecule R in the reagent A can interact with the biomolecule L in the reagent B to cause a phase transition to produce phase transition droplets, wherein, one of the biomolecule R and the biomolecule L is a multivalent molecule and one is the corresponding multivalent ligand, the biomolecule X in the reagent A can recruit the reagent C into phase transition droplets through interacting with the biomolecule X_{L} in the reagent C, and the fluorescence signal of the fluorescent reporter group JIA connected to the biomolecule X_{L} in the reagent C in the phase transition droplets is used to determine whether the biomolecule X and the biomolecule X_{L} interact. In addition, by adding regulatory factors to the reaction system, the influence of regulatory factors on the interaction between the biomolecule X and the biomolecule X_{L} can be determined, and furthermore, the screening of the regulatory factors for interaction between biomolecules can be conducted.

The present invention converts microscopic protein interactions and their biochemical processes such as interaction regulation into intuitive fluorescence signal intensity changes with high visibility; the operation process is simple and easy and the cost is low; since the concentration of phase transition protein is close to the protein concentration in human body, it can simulate the real life environment to the most extent; in addition, this method has the characteristics of high sensitivity and wide applicability, which provides a new idea and method for high-throughput screening of regulatory factors for protein interaction.

Besides, in the present invention, the reagent sets for detecting the interaction between the post-translationally modified protein and its ligand, and the multivalent recruitment systems based on the above reagent sets utilize the phase transition mechanism to realize the high-level enrichment of the proteins and their ligands into the phase transition droplets, which could significantly amplifies the original weak interaction signal and thus makes it easy to detect. The reagent sets and the multivalent recruitment systems of the present invention can be used to detect interactions between proteins and their ligands, especially weak interactions, and can also be used to identify whether a protein has a certain kind of enzyme activity involved in a the post-translational modification, such as the kinase activity corresponding to the phosphorylation and the methyltransferase activity corresponding to the methylation, and can also be used to identify regulators that can regulate the above enzyme activity.

Further, the method for detecting intracellular biomolecular interactions of the present invention can be used to detect intracellular biomolecular interactions, and this method can be used to further screen regulatory factors that affect the interaction between biomolecule pairs known to have interactions. The method of the present invention has the advantages of simple operation, high sensitivity, low cost and wide applicability, and is suitable for screening regulators of signal pathways, and can also be applied to high-throughput screening for regulatory factors for interactions between biomolecules.

What is claimed is:

1. A reagent set, which is reagent set I or reagent set II, as follows:
reagent set I, consisting of three reagents named A, B and C, respectively;
the reagent A is formed by connecting a biomolecule named R and a biomolecule named X;
the reagent B contains a biomolecule named L;
the biomolecule R and the biomolecule L are the same or different and there is an interaction between the two, and a phase transition occurs after the biomolecule R and the biomolecule L interact;
the reagent C is formed by connecting a reporter group named JIA with a biomolecule named X_{L};
the biomolecule X is a protein, a nucleic acid, or a polysaccharide; the biomolecule X_{L} is a protein, a nucleic acid, or a polysaccharide;
reagent set II, consisting of four reagents named A, B, E and D, respectively;
the reagent A is formed by connecting a biomolecule named R and a biomolecule named X;
the biomolecule X is a protein;
the reagent B contains a biomolecule named L;
the biomolecule R and the biomolecule L are the same or different and there is an interaction between the two, and a phase transition occurs when the biomolecule R and the biomolecule L interact;
the reagent E is a polymer formed by E monomers, and the E monomer is the following c1) or c2):
c1) a molecule obtained by connecting a monomer named mc, a reporter group named JIA, and a biomolecule named Y_{C}, two or more mc monomers can form a polymer;
c2) a molecule obtained by ligating a tag to c1);
the reagent D is formed by connecting a modified protein named X_{L} and a biomolecule named Y_{D};
there is an interaction between the biomolecule Y_{C} and the biomolecule Y_{D}.

2. The reagent set according to claim 1, wherein in the reagent set I, it is unknown whether there is an interaction between the biomolecule X and the biomolecule X_{L}, and the reagent set is used to detect or assist in detecting whether there is an interaction between the biomolecule X and the biomolecule X_{L}.

3. The reagent set according to claim 1, wherein in the reagent set I, there is an interaction between the biomolecule X and the biomolecule X_{L}, and the reagent set is used to identify or assist in identifying a regulatory factor for the interaction between the biomolecule X and the biomolecule X_{L}.

4. (canceled)

5. The reagent set according to claim 1, wherein in the reagent set II, both the biomolecule Y_{C} and the biomolecule Y_{D} are proteins.

6. The reagent set according to claim 1, wherein in the reagent set II, the biomolecule Y_{C} is the following Y11), Y12) or Y13):
Y11) a protein having the amino acid sequence as shown in positions 362-465 of SEQ ID NO: 19;
Y12) a protein obtained by substitution and/or deletion and/or addition of one or more amino acid residues in the amino acid sequence as shown in positions 362-465 of SEQ ID NO: 19 in the Sequence Listing and having the same function;
Y13) a fusion protein obtained by ligating tag(s) to the N-terminus or/and C-terminus of Y11) or Y12); and/or,
the biomolecule Y_{D} is the following Y21), Y22) or Y23):
Y21) a protein having the amino acid sequence as shown in positions 22-29 of SEQ ID NO: 23;
Y22) a protein obtained by substitution and/or deletion and/or addition of one or more amino acid residues in the amino acid sequence as shown in positions 22-29 of SEQ ID NO: 23 in the Sequence Listing and having the same function;
Y23) a fusion protein obtained by ligating tag(s) to the N-terminus or/and C-terminus of Y21) or Y22).

7. The reagent set according to claim 1, wherein in both reagent set I and reagent set II, the biomolecule R contains a binding region named binding region 1; and the biomolecule L contains a binding region named binding region 2; and the interaction between the biomolecule R and the biomolecule L is realized by the binding region 1 and the binding region 2, and both the number of the binding region 1 in the biomolecule R and the number of the binding region 2 in the biomolecule L is greater than or equal to 2.

8. The reagent set according to claim 1, wherein the biomolecule R is a protein, a nucleic acid, or a polysaccharide; and/or, the biomolecule L is a protein, a nucleic acid, or a polysaccharide.

9. The reagent set according to claim 1, wherein in both reagent set I and reagent set II, the reagent A is further connected with a reporting group named YI; and/or,
the reagent B is further connected with a report Group named BING.

10. The reagent set according to claim 9, wherein in both reagent set I and reagent set II, the report Group YI and the report Group BING are the same or different; and/or,
the report Group JIA is different from the report Group YI and the report Group BING.

11. (canceled)

12. The reagent set according to claim 1, wherein in both reagent set I and reagent set II, the ratio of the number of the biomolecule X to the number of the biomolecule R in the reagent A is an integer greater than or equal to 1.

13. The reagent set according to claim 1, wherein in both reagent set I and reagent set II, the biomolecule R is a polymer formed by R monomers, and each R monomer contains a monomer named mr, and two or more mr monomers can form a polymer; and/or,
the biomolecule L is a polymer formed by L monomers, and each monomer L contains a monomer named ml, and two or more ml monomers can form a polymer;
the mc monomer, the mr monomer and the ml monomer are the same or at least two of them are the same or they are different from each other.

14. The reagent set according to claim 13, wherein in both reagent set I and reagent set II, at least one monomer in the biomolecule R contains the binding region 1; and/or,
at least one monomer in the biomolecule L contains the binding region 2.

15. The reagent set according to claim 13, wherein in both reagent set I and reagent set II, each R monomer contains the mr monomer and the binding region 1; and/or,
each L monomer contains the ml monomer and the binding region 2.

16. The reagent set according to claim 15, wherein in both reagent set I and reagent set II, in the R monomer, the mr monomer and the binding region 1 or a biomolecule containing the binding region 1 are connected through a linking region or a chemical bond; and/or,
in the L monomer, the monomer ml and the binding region 2 or a biomolecule containing the binding region 2 are connected through a linking region or a chemical bond.

17. The reagent set according to claim 16, wherein in both reagent set I and reagent set II, each R monomer further contains the reporter group YI; and/or,
each L monomer further contains the reporter group BING.

18. The reagent set according to claim 17, wherein in both reagent set I and reagent set II, in the R monomer, the mr monomer, the reporter group YI, and the binding region 1 or a biomolecule containing the binding region 1 are connected through a linking region or a chemical bond; and/or,
in the L monomer, the ml monomer, the reporter group BING, and the binding region 2 or a biomolecule containing the binding region 2 are connected through a linking region or a chemical bond.

19. The reagent set according to claim 18, wherein in both reagent set I and reagent set II, all the R monomers are the same, all the L monomers are the same, and all the E monomers are the same; and/or,
Both the mr monomer and the ml monomer are yeast protein SmF; and/or, the mc is monomer is Bacillus subtilis protein Hfq; and/or,
the binding region 1 is a region in SH3 as shown in positions 364-431 of SEQ ID NO: 1 that binds to PRMH as shown in positions 366-380 of SEQ ID NO: 5; the binding region 2 is a region in PRMH as shown in positions 366-380 of SEQ ID NO: 5 that binds to SH3 as shown in positions 364-431 of SEQ ID NO: 1; and/or,
the linking region is (Gly-Gly-Ser)ₙ or a polypeptide containing (Gly-Gly-Ser)ₙ, and n is a natural number greater than or equal to 2; and/or,
the reporter group JIA is a red fluorescent protein; and/or,
the reporter group YI and the reporter group BING are green fluorescent protein.

20. The reagent set according to claim 19, wherein in both reagent set I and reagent set II, both the mr monomer and the ml monomer are yeast SmF as shown in positions 17-102 of SEQ ID NO: 1; and/or,
the mc monomer is Hfq as shown in positions 17-94 of SEQ ID NO: 19; and/or,
the biomolecule containing the binding region 1 is SH3 as shown in positions 364-431 of SEQ ID NO: 1; and/or,
the biomolecule containing the binding region 2 is PRMH as shown in positions 366-380 of SEQ ID NO: 5.

21-44. (canceled)

45. The reagent set according to claim 20, wherein in both reagent set I and reagent set II, the R monomer is the following H1) or H2) or H3):
H1) a protein having the amino acid sequence as shown in positions 17-431 of SEQ ID NO: 1;
H2) a protein obtained by substitution and/or deletion and/or addition of one or more amino acid residues in the amino acid sequence as shown in positions 17-341 of SEQ ID NO: 1 in the Sequence Listing and having the same function;
H3) a fusion protein obtained by ligating tag(s) to the N-terminus or/and C-terminus of H1) or H2); and/or,
the L monomer is the following I1) or I2) or I3):
I1) a protein having the amino acid sequence as shown in positions 17-380 of SEQ ID NO: 5;
I2) a protein obtained by substitution and/or deletion and/or addition of one or more amino acid residues in the amino acid sequence as shown in positions 17-380 of SEQ ID NO: 5 in the Sequence Listing and having the same function;
I3) a fusion protein obtained by ligating tag(s) to the N-terminus or/and C-terminus of I1) or I2); and/or
the E monomer is the following J1) or J2) or J3):
J1) a protein having the amino acid sequence as shown in positions 17-465 of SEQ ID NO: 19;
J2) a protein obtained by substitution and/or deletion and/or addition of one or more amino acid residues in the amino acid sequence as shown in positions 17-465 of SEQ ID NO: 19 in the Sequence Listing and having the same function;
J3) a fusion protein obtained by ligating tag(s) to the N-terminus or/and C-terminus of J1) or J2).

46. A reagent set, consisting of the following X1) and X2) or consisting of the following X1), X2), X3) and X4):
X1) a biological material related to the R monomer in claim 1, which is any one of the following X11) to X14):
X11) a nucleic acid molecule encoding the R monomer in claim 1;
X12) an expression cassette containing the nucleic acid molecule of X11);
X13) a recombinant vector containing the nucleic acid molecule of X11), or a recombinant vector containing the expression cassette of X12);
X14) a recombinant microorganism containing the nucleic acid molecule of X11), or a recombinant microorganism containing the expression cassette of X12), or a recombinant microorganism containing the recombinant vector of X13);
X2) a biological material related to the L monomer in claim 1, which is any one of the following X21) to X24):
X21) a nucleic acid molecule encoding the L monomer in claim 1;
X22) an expression cassette containing the nucleic acid molecule of X21);
X23) a recombinant vector containing the nucleic acid molecule of X21), or a recombinant vector containing the expression cassette of X22);
X24) a recombinant microorganism containing the nucleic acid molecule of X21), or a recombinant microorganism containing the expression cassette of X22), or a recombinant microorganism containing the recombinant vector of X23).
X3) a biological material related to the E monomer in claim 1, which is any one of the following X31) to X34):
X31) a nucleic acid molecule encoding the E monomer in claim 1;
X32) an expression cassette containing the nucleic acid molecule of X31);
X33) a recombinant vector containing the nucleic acid molecule of X31), or a recombinant vector containing the expression cassette of X32);
X34) a recombinant microorganism containing the nucleic acid molecule of X31), or a recombinant microorganism containing the expression cassette of X32), or a recombinant microorganism containing the recombinant vector of X33).
X4) a biological material related to the biomolecule Y_{D} in claim 1, which is any one of the following X41) to X44):
X41) a nucleic acid molecule encoding the biomolecule Y_{D} in claim 1;
X42) an expression cassette containing the nucleic acid molecule of X41);
X43) a recombinant vector containing the nucleic acid molecule of X41), or a recombinant vector containing the expression cassette of X42);
X44) a recombinant microorganism containing the nucleic acid molecule of X41), or a recombinant microorganism containing the expression cassette of X42), or a recombinant microorganism containing the recombinant vector of X43).

47. The reagent set according to claim 46, wherein the nucleic acid molecule of X11) is the following x11) or x12) or x13):
x11) a cDNA molecule or a DNA molecule having the encoding sequence as shown in positions 62-1306 of SEQ ID NO: 2 in the Sequence Listing;
x12) a cDNA molecule or a genomic DNA molecule having 75% or more identity with the nucleotide sequence defined by x11) and encoding the R monomer is yeast protein SmF;
x13) a cDNA molecule or a genomic DNA molecule hybridizing to the nucleotide sequence defined by x11) under stringent conditions and encoding the R monomer is yeast protein SmF; and/or,
the nucleic acid molecule of X21) is the following x21) or x22) or x23):
x21) a cDNA molecule or a DNA molecule having the encoding sequence as shown in positions 62-1153 of SEQ ID NO: 6 in the Sequence Listing;
x22) a cDNA molecule or a genomic DNA molecule having 75% or more identity with the nucleotide sequence defined by x21) and encoding the L monomer is yeast protein SmF;
x23) a cDNA molecule or a genomic DNA molecule hybridizing to the nucleotide sequence defined by x21) under stringent conditions and encoding the L monomer is yeast protein SmF; and/or
the nucleic acid molecule of X31) is the following x31) or x32) or x33):
x31) a cDNA molecule or a DNA molecule having the encoding sequence as shown in positions 51-1400 of SEQ ID NO: 20 in the Sequence Listing;
x32) a cDNA molecule or a genomic DNA molecule having 75% or more identity with the nucleotide sequence defined by x31) and encoding the E monomer, and the E monomer is the following c1) or c2):
c1) a molecule obtained by connecting a monomer named mc, a reporter group named JIA, and a biomolecule named Y_{C}, two or more mc monomers can form a polymer;
c2) a molecule obtained by ligating a tag to c1);
x33) a cDNA molecule or a genomic DNA molecule hybridizing to the nucleotide sequence defined by x31) under stringent conditions and encoding the E monomer, and the E monomer is the following c1) or c2);
c1) a molecule obtained by connecting a monomer named mc, a reporter group named JIA, and a biomolecule named Y_{C}, two or more mc monomers can form a polymer;
c2) a molecule obtained by ligating a tag to c1).

48. The reagent set according to claim 1, wherein in the reagent set II, the modification is a protein post-translational modification or a de-modification of protein post-translational modification;
further, the protein post-translational modification is methylation, acetylation, phosphorylation, ubiquitination or glycosylation modification;
the de-modification of protein post-translational modification is demethylation, deacetylation, dephosphorylation, deubiquitination or deglycosylation.

49. A method for detecting whether there is an interaction between biomolecules, wherein the biomolecules are two biomolecules named X and X_{L}, respectively, and the method comprises the following steps:
a solution to be tested is obtained by mixing solution A, solution B and solution C; the solution A is a solution containing the reagent A in claim 1; the solution B is a solution containing the reagent B in claim 1; the solution C is a solution containing the reagent C in claim 1; the biomolecule R in the reagent A and the biomolecule L in the reagent B in the solution to be tested interact to produce phase transition droplets; according to whether there is a signal of the reporter group JIA in the phase transition droplets in the solution to be tested, the interaction between the biomolecule X and the biomolecule X_{L} is determined.

50. The method according to claim 49, wherein the biomolecule X_{L} is a modified protein, and the biomolecule X is a protein, and the method comprises the following steps:
a solution to be tested is obtained by mixing solution A, solution B, solution E and solution D; the solution E is a solution containing the reagent E in claim 1; the solution D is a solution containing the reagent D in claim 1; the biomolecule R in the reagent A and the biomolecule L in the reagent B in the solution to be tested interact to produce phase transition droplets; according to whether there is a signal of the reporter group JIA in the phase transition droplets in the solution to be tested, the interaction between the biomolecule X and the biomolecule X_{L} is determined.

51. The method according to claim 50, wherein the modification is a protein post-translational modification or a de-modification of protein post-translational modification;
further, the protein post-translational modification is methylation, acetylation, phosphorylation, ubiquitination or glycosylation modification;
the de-modification of protein post-translational modification is demethylation, deacetylation, dephosphorylation, deubiquitination or deglycosylation.

52. The method according to claim 49, wherein the method is used for identifying a regulatory factor between biomolecules, wherein the biomolecules are two biomolecules named X and X_{L}, respectively and there is an interaction between the biomolecule X and the biomolecule X_{L}, and the method comprises the following steps:
a solution to be tested is obtained by mixing solution A, solution B, solution C and a regulatory factor to be tested; a control solution is obtained by mixing the solution A, the solution B and the solution C; in the solution to be tested and the control solution, the biomolecule R in the solution A and the biomolecule L in the solution B interact to produce phase transition droplets; by comparing the signal intensity of the reporter group JIA in the phase transition droplets in the solution to be tested with that in the control solution, it is determined whether the regulatory factor to be tested has a regulatory effect on the interaction between the biomolecule X and the biomolecule X_{L};
the solution A is a solution containing the reagent A in claim 1; the solution B is a solution containing the reagent B in claim 1; the solution C is a solution containing the reagent C in claim 1.

53. The method according to claim 52, wherein the method is used for detecting whether a protein has an enzyme activity involved in a protein post-translational modification.

54. A method for detecting the interaction between biomolecules in a cell, the biomolecules to be tested are named X and X_{L}, the biomolecule X is a protein, a nucleic acid or a polysaccharide, and the biomolecule X_{L} is a protein, a nucleic acid or a polysaccharide, and the method comprises U1) and U2):
U1) connecting a biomolecule named R and the biomolecule X to obtain a recombinant molecule named R—X; the biomolecule R containing intrinsically disordered proteins/regions; connecting the biomolecule X_{L} and a reporter group named J to obtain a recombinant molecule named X_{L}-J;
U2) introducing the recombinant molecule R—X and the recombinant molecule X_{L}-J into a biological cell to obtain a recombinant cell, and detecting whether the signal of the reporter group J in the recombinant cell is accumulated in a second phase formed by the intrinsically disordered proteins/regions to determine whether there is an interaction between the biomolecule X and the biomolecule X_{L}; if the signal of the reporter group J is accumulated in the second phase, the biomolecule X and the biomolecule X_{L} have an interaction or are supposed to have an interaction; if the signal of the reporter group J is not accumulated in the second phase, the biomolecule X and the biomolecule X_{L} have no interaction or are supposed to have no interaction.

55. The method according to claim 54, wherein the method is used for identifying regulatory factors for interactions between biomolecules in a cell, the biomolecules to be tested are named X and X_{L}, the biomolecule X is a protein, a nucleic acid or a polysaccharide, and the biomolecule X is a protein, a nucleic acid or a polysaccharide, and the method comprises V1) and V2):
V1) connecting a biomolecule named R and the biomolecule X to obtain a recombinant molecule named R—X; the biomolecule R containing intrinsically disordered proteins/regions; connecting the biomolecule X_{L} and a reporter group named J to obtain a recombinant molecule named X_{L}-J;
V2) introducing the recombinant molecule R—X and the recombinant molecule X_{L}-J into a biological cell to obtain a recombinant cell; culturing the recombinant cell and adding a regulatory factor to be tested to the culture system of the recombinant cell to obtain a system to be tested; culturing the recombinant cell to obtain a control system; then detecting the signal intensity of the reporter group J in the recombinant cell in a second phase formed by the intrinsically disordered proteins/regions in the system to be tested and the control system to determine whether the regulatory factor to be tested has a regulatory effect on the interaction between the biomolecule X and the biomolecule X_{L}: if the signal of the reporter group J in the second phase in the system to be tested is stronger than the signal of the reporter group J in the second phase in the control system, the regulatory factor to be tested has or is supposed to have a promoting effect on the interaction between the biomolecule X and the biomolecule X_{L}; if the signal intensity of the reporter group J in the second phase in the system to be tested is the same as the signal intensity of the reporter group J in the second phase in the control system, the regulatory factor to be tested has or is supposed to have no regulatory effect on the interaction between the biomolecule X and the biomolecule X_{L}; if the signal intensity of the reporter group J in the second phase in the system to be tested is weaker than the signal of the reporter group J in the second phase in the control system, the regulatory factor to be tested has or is supposed to have an inhibitory effect on the interaction between the biomolecule X and the biomolecule X_{L}.

56. The method according to claim 54, wherein the biomolecule R can further contain a reporter group named K, and the reporter group K is different from the reporter group J.

57. The method according to claim 54, wherein the intrinsically disordered protein/region is the following H1) or H2) or H3):
H1) a protein having the amino acid sequence as shown in positions 258-772 of SEQ ID NO: 24;
H2) a protein obtained by substitution and/or deletion and/or addition of one or more amino acid residues in the amino acid sequence as shown in positions 258-772 of SEQ ID NO: 24 in the Sequence Listing and having the same function;
H3) a fusion protein obtained by ligating tag(s) to the N-terminus or/and C-terminus of H1) or H2).

58. The method according to claim 54, wherein the reporter group K in the biomolecule R and the intrinsically disordered proteins/regions are connected through a linking region or a chemical bond;
the biomolecule X_{L} and the reporter group J in the recombinant molecule X_{L}-J are connected through a linking region or a chemical bond; and/or,
the biomolecule R and the biomolecule X in the recombinant molecule R—X are connected through a linking region or a chemical bond.

59. The method according to claim 59, wherein the linking region is (Gly-Gly-Ser)ₙ or a polypeptide containing (Gly-Gly-Ser)ₙ, and n is a natural number greater than or equal to 2.

60. The method according to claim 54, wherein the biomolecule R is the following I1) or I2) or I3) or I4):
I1) a protein having the amino acid sequence as shown in positions 1-772 of SEQ ID NO: 24;
I2) a protein having the amino acid sequence as shown in positions 1-784 of SEQ ID NO: 24;
I3) a protein obtained by substitution and/or deletion and/or addition of one or more amino acid residues in the amino acid sequence as shown in positions 1-772 or 1-784 of SEQ ID NO: 24 in the Sequence Listing and having the same function;
I4) a fusion protein obtained by ligating tag(s) to the N-terminus or/and C-terminus of I1) or I2) or I3).

61. The use according to claim 54, wherein the biological cell is an animal cell, a plant cell, or a microbial cell.

62. The biomolecule R in claim 54.

63. A biological material related to the biomolecule R in claim 63, and the biological material is any one of the following M1) to M4):
M1) a nucleic acid molecule encoding the biomolecule R;
M2) an expression cassette containing the nucleic acid molecule of M1);
M3) a recombinant vector containing the nucleic acid molecule of M1), or a recombinant vector containing the expression cassette of M2);
M4) a recombinant microorganism containing the nucleic acid molecule of M1), or a recombinant microorganism containing the expression cassette of M2), or a recombinant microorganism containing the recombinant vector of M3).

64. The biological material according to claim 54, wherein the nucleic acid molecule of M1) is any one of the following m1)-m8):
m1) a cDNA molecule or a DNA molecule having the encoding sequence as shown in positions 780-2324 of SEQ ID NO: 25 in the Sequence Listing;
m2) a cDNA molecule or a DNA molecule having the encoding sequence as shown in positions 738-2324 of SEQ ID NO: 25 in the Sequence Listing;
m3) a cDNA molecule or a DNA molecule having the encoding sequence as shown in positions 9-2324 of SEQ ID NO: 25 in the Sequence Listing;
m4) a cDNA molecule or a DNA molecule having the encoding sequence as shown in positions 780-2360 of SEQ ID NO: 25 in the Sequence Listing;
m5) a cDNA molecule or a DNA molecule having the encoding sequence as shown in positions 738-2360 of SEQ ID NO: 25 in the Sequence Listing;
m6) a cDNA molecule or a DNA molecule having the encoding sequence as shown in positions 9-2360 of SEQ ID NO: 25 in the Sequence Listing;
m7) a cDNA molecule or a DNA molecule having 75% or more identity with the nucleotide sequence defined by m1) or m2) or m3) or m4) or m5) or m6) and encoding the biomolecule R in claim 54;
m8) a cDNA molecule or a DNA molecule hybridizing to the nucleotide sequence defined by m1) or m2) or m3) or m4) or m5) or m6) under stringent conditions and encoding the biomolecule R in claim 54.


SEQUENCE LISTING (30 sequences)

SEQ ID NO: 1 - protein, 446 aa, Artificial Sequence
  Synthesized
MKHHHHHHENLYFQGGMSESSDISAMQPVNPKPFLKGLVNHRVGVKLKFNSTEYRGTLVSTDNYFNLQLNEAEEFVAGVSHGTLGEIFIRSNNVLYIRELPNGGSGGSMKVSKGEELFTGVVPILVELDGDVNGHKFSVRGEGEGDATNGKLTLKFICTTGKLPVPWPTLVTTLTYGVQCFSRYPDYMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITLGMDELYKTMKGGSGGSGGSGGSMSGHMDLNMPAYVKFNYMAEREDELSLIKGTKVIVMEKSSDGWWRGSYNGQVGWFPSNYVTEEGDSPLGGSGGSGGSGGSSMG

SEQ ID NO: 2 - DNA, 1374 bp, Artificial Sequence
  Synthesized
aaggagatataccatgaaacatcatcatcatcatcacgaaaacctgtattttcagggcggcatgagcgaaagcagcgatattagcgcgatgcagccggtgaacccgaaaccgtttctgaaaggcctggtgaaccatcgcgtgggcgtgaaactgaaatttaacagcaccgaatatcgcggcaccctggtgagcaccgataactattttaacctgcaactgaacgaagcggaagaatttgtggcgggcgtgagccacggcaccctgggcgaaatttttattcgcagcaacaacgtgctgtatattcgcgaactgccgaacggcggttccggcggttccatgaaagtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgactacatgaagcagcacgacttcttcaagtccgccatgcccgaaggctacgtccaggagcgcaccatctccttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcacggccgacaagcagaagaacggcatcaaggcgaacttcaagatccgccacaacgtcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagaccatgaaaggcggtagcggtggcagcggtggtagcggcggctccatgagcggccatatggacctcaacatgcccgcttatgtgaaatttaactacatggctgagagagaggatgaattatcattgataaaggggacaaaggtgatcgtcatggagaaaagcagtgatgggtggtggcgtggtagctacaatggacaagttggatggttcccttcaaactatgtaactgaagaaggtgacagtcctttgggtggcagtggcggtagcggtggcagcggtggcagctccatgggctaactcgagtctggtaaagaaac

SEQ ID NO: 3 - protein, 15 aa, Artificial Sequence
  Synthesized
SQETFSDLWKLLPEN

SEQ ID NO: 4 - DNA, 70 bp, Artificial Sequence
  Synthesized
ggaaccatgggcagccaggaaacctttagcgatctgtggaaactgctgccggaaaactaactcgagaagg

SEQ ID NO: 5 - protein, 380 aa, Artificial Sequence
  Synthesized
MKHHHHHHENLYFQGGMSESSDISAMQPVNPKPFLKGLVNHRVGVKLKFNSTEYRGTLVSTDNYFNLQLNEAEEFVAGVSHGTLGEIFIRSNNVLYIRELPNGGSGGSMKVSKGEELFTGVVPILVELDGDVNGHKFSVRGEGEGDATNGKLTLKFICTTGKLPVPWPTLVTTLTYGVQCFSRYPDYMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITLGMDELYKTMKGGSGGSGGSGGSMSSKKTPPPVPPRTTSK

SEQ ID NO: 6 - DNA, 1183 bp, Artificial Sequence
  Synthesized
aaggagatataccatgaaacatcatcatcatcatcacgaaaacctgtattttcagggcggcatgagcgaaagcagcgatattagcgcgatgcagccggtgaacccgaaaccgtttctgaaaggcctggtgaaccatcgcgtgggcgtgaaactgaaatttaacagcaccgaatatcgcggcaccctggtgagcaccgataactattttaacctgcaactgaacgaagcggaagaatttgtggcgggcgtgagccacggcaccctgggcgaaatttttattcgcagcaacaacgtgctgtatattcgcgaactgccgaacggcggttccggcggttccatgaaagtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgactacatgaagcagcacgacttcttcaagtccgccatgcccgaaggctacgtccaggagcgcaccatctccttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcacggccgacaagcagaagaacggcatcaaggcgaacttcaagatccgccacaacgtcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagaccatgaaaggcggtagcggtggcagcggtggtagcggcggctccatgagcagcaaaaaaaccccgccgccggtgccgccgcgcaccaccagcaaataactcgagtctggtaaagaaaccgctgct

SEQ ID NO: 7 - protein, 270 aa, Artificial Sequence
  Synthesized
MKHHHHHHENLYFQGGMKVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKTMKGGSGGSGGSGGSMG

SEQ ID NO: 8 - DNA, 846 bp, Artificial Sequence
  Synthesized
aggagatataccatgaaacatcatcatcatcatcacgaaaacctgtattttcagggcggcatgaaagtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgtggcaccaacttcccctccgacggccccgtaatgcagaagaagacaatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaccatgaaaggcggtagcggtggcagcggtggtagcggcggctccatgggctaactcgagtctggtaaagaaacc

SEQ ID NO: 9 - protein, 103 aa, Artificial Sequence
  Synthesized
MTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVN

SEQ ID NO: 10 - DNA, 328 bp, Artificial Sequence
  Synthesized
ggaatcatgactgatggtgctgtaaccaccagccagattccggcgagcgaacaggaaaccctggtgcgcccgaaaccgctgctgctgaaactgctgaaaagcgtgggcgcgcagaaagatacctataccatgaaagaagtgctgttttatctgggccagtatattatgaccaaacgcctgtatgatgaaaaacagcagcatattgtgtattgcagcaacgatctgctgggcgatctgtttggcgtgccgagctttagcgtgaaagaacatcgcaaaatttataccatgatttatcgcaacctggtggtggtgaactaactcgagaagg

SEQ ID NO: 11 - protein, 109 aa, Artificial Sequence
  Synthesized
MGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 12 - DNA, 338 bp, Artificial Sequence
  Synthesized
ccatggggggcgtgcaggtggaaaccattagcccgggcgatggccgcacctttccgaaacgcggccagacctgcgtggtgcattataccggtatgctggaagatggcaaaaaatttgatagcagccgcgatcgcaacaaaccgtttaaatttatgctgggcaaacaggaagtgattcgcggctgggaagaaggcgtggcgcagatgagcgtgggccagcgcgcgaaactgaccattagcccggattatgcgtatggcgcgaccggccatccgggcattattccgccgcacgcgaccctggtgtttgatgtggaactgctgaaactggaataactcgag

SEQ ID NO: 13 - protein, 96 aa, Artificial Sequence
  Synthesized
MERVAILWHEMWHEGLEEASRLYFGERNVKGMFEVLEPLHAMMERGPQTLKETSFNQAYGRDLMEAQEWCRKYMKSGNVKDLTQAWDLYYHVFRRI

SEQ ID NO: 14 - DNA, 299 bp, Artificial Sequence
  Synthesized
ccatggaacgcgtggcgattctgtggcatgaaatgtggcatgaaggcctggaagaagcgagccgcctgtattttggcgaacgcaacgtgaaaggcatgtttgaagtgctggaaccgctgcacgcgatgatggaacgcggcccgcagaccctgaaagaaaccagctttaaccaggcgtatggccgcgatctgatggaagcgcaggaatggtgccgcaaatatatgaaaagcggcaacgtgaaagatctgacccaggcgtgggatctgtattatcatgtgtttcgccgcatttaactcgag

SEQ ID NO: 15 - protein, 8 aa, Artificial Sequence
  Synthesized
MGKKETPV

SEQ ID NO: 16 - DNA, 35 bp, Artificial Sequence
  Synthesized
ccatgggcaagaaagaaaccccggtgtaactcgag

SEQ ID NO: 17 - protein, 65 aa, Artificial Sequence
  Synthesized
EEYVVEKVLDRRVVKGKVEYLLKWKGFSDEDNTWEPEENLDCPDLIAEFLQSQKTAHETDKSEGG

SEQ ID NO: 18 - DNA, 226 bp, Artificial Sequence
  Synthesized
ccatgggggaggaatatgtggtggaaaaagttctcgaccgtcgagtggtaaagggcaaagtggagtacctcctaaagtggaagggattctcagatgaggacaacacatgggagccagaagagaacctggattgccccgacctcattgctgagtttctgcagtcacagaaaacagcacatgagacagataaatcagagggaggctaactcgagtctggtaaagaaac

SEQ ID NO: 19 - protein, 465 aa, Artificial Sequence
  Synthesized
MKHHHHHHENLYFQGGGPLGSMKPINIQDQFLNQIRKENTYVTVFLLNGFQLRGQVKGFDNFTVLLESEGKQQLIYKHAISTFAPQKNVQLELEGGSGGSMKVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKTMKGGSGGSGGSGGSMGGSGGSGSPEFLGEEDIPREPRRIVIHRGSTGLGFNIVGGEDGEGIFISFILAGGPADLSGELRKGDQILSVNGVDLRNASHEQAAIALKNAGQTVTIIAQYKPEEYSRFEA

SEQ ID NO: 20 - DNA, 1406 bp, Artificial Sequence
  Synthesized
ccatgaaacatcatcatcatcatcacgaaaacctgtattttcagggcggcggcccgctgggtagcatgaaaccgattaacattcaggatcagtttctgaaccagattcgcaaagaaaacacctatgtgaccgtgtttctgctgaacggctttcagctgcgcggccaggtgaaaggctttgataactttaccgtgctgctggaaagcgaaggcaaacagcagctgatttataaacatgcgattagcacctttgcgccgcagaaaaacgtgcagctggaactggaaggcggttccggcggttccatgaaagtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgtggcaccaacttcccctccgacggccccgtaatgcagaagaagacaatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaccatgaaaggcggtagcggtggcagcggtggtagcggcggctccatgggtggcagcggcggtagcggatccccggaattcctgggggaggaagacattccccgggaaccaaggcggatcgtgatccatcggggctccaccggcctgggcttcaacattgtgggcggcgaggatggtgaaggcatcttcatctccttcatccttgctgggggtccagccgacctcagtggggagctacggaagggggaccagatcctgtcggtcaatggtgttgacctccgcaatgccagtcacgaacaggctgccattgccctgaagaatgcgggtcagacggtcacgatcatcgctcagtataaaccagaagagtatagtcgattcgaggcgtaactcgag

SEQ ID NO: 21 - protein, 374 aa, Artificial Sequence
  Synthesized
MKHHHHHHENLYFQGGMKVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKTMKGGSGGSGGSGGSMGPEFLGEEDIPREPRRIVIHRGSTGLGFNIVGGEDGEGIFISFILAGGPADLSGELRKGDQILSVNGVDLRNASHEQAAIALKNAGQTVTIIAQYKPEEYSRFEA

SEQ ID NO: 22 - DNA, 1158 bp, Artificial Sequence
  Synthesized
aggagatataccatgaaacatcatcatcatcatcacgaaaacctgtattttcagggcggcatgaaagtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgtggcaccaacttcccctccgacggccccgtaatgcagaagaagacaatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaccatgaaaggcggtagcggtggcagcggtggtagcggcggctccatgggcccggaattcctgggggaggaagacattccccgggaaccaaggcggatcgtgatccatcggggctccaccggcctgggcttcaacattgtgggcggcgaggatggtgaaggcatcttcatctccttcatccttgctgggggtccagccgacctcagtggggagctacggaagggggaccagatcctgtcggtcaatggtgttgacctccgcaatgccagtcacgaacaggctgccattgccctgaagaatgcgggtcagacggtcacgatcatcgctcagtataaaccagaagagtatagtcgattcgaggcgtaactcgagtctggtaaagaaacc

SEQ ID NO: 23 - protein, 29 aa, Artificial Sequence
  Synthesized
ARTKQTARGGSGGSGGSWGGSKKETPVAV

SEQ ID NO: 24 - protein, 784 aa, Artificial Sequence
  Synthesized
MKVSKGEELFTGVVPILVELDGDVNGHKFSVRGEGEGDATNGKLTLKFICTTGKLPVPWPTLVTTLTYGVQCFSRYPDYMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITLGMDELYKTMKGGSGGSGGSGGSLEMFNKSFGTPFGGGTGGFGTTSTFGQNTGFGTTSGGAFGTSAFGSSNNTGGLFGNSQTKPGGLFGTSSFSQPATSTSTGFGFGTSTGTANTLFGTASTGTSLFSSQNNAFAQNKPTGFGNFGTSTSSGGLFGTTNTTSNPFGSTSGSLFGPSSFTAAPTGTTIKFNPPTGTDTMVKAGVSTNISTKHQCITAMKEYESKSLEELRLEDYQANRKGPQNQVGAGTTTGLFGSSPATSSATGLFSSSTTNSGFAYGQNKTAFGTSTTGFGTNPGGLFGQQNQQTTSLFSKPFGQATTTQNTGFSFGNTSTIGQPSTNTMGLFGVTQASQPGGLFGTATNTSTGTAFGTGTGLFGQTNTGFGAVGSTLFGNNKLTTFGSSTTSAPSFGTTSGGLFGFGTNTSGNSIFGSKPAPGTLGTGLGAGFGTALGAGQASLFGNNQPKIGGPLGTGAFGAPGFNTTTATLGFGAPQAPVALTDPNASAAQQAVLQQHINSLTYSPFGDSPLFRNPMSDPKKKEEEGGSGGSGGSGGS

SEQ ID NO: 25 - DNA, 2369 bp, Artificial Sequence
  Synthesized
gcggccgcatgaaagtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgactacatgaagcagcacgacttcttcaagtccgccatgcccgaaggctacgtccaggagcgcaccatctccttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcacggccgacaagcagaagaacggcatcaaggcgaacttcaagatccgccacaacgtcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagaccatgaaaggcggtagcggtggcagcggtggtagcggcggctccctcgagatgtttaacaaatcatttggaacaccctttgggggtggcacaggtggctttggcacaacttcaacatttggacagaatactggctttggcactactagtggaggggcatttggaacatctgcatttggttctagcaacaatactggaggcctctttggaaattcacagactaaaccaggaggattgtttggaaccagttcatttagccagccagctacctccacaagcactggctttgggtttggtacgtcaacaggaacagcaaataccttgtttggaactgcaagcacagggaccagtctcttctcatcccaaaacaatgcctttgcacaaaataaaccaactggctttggcaattttggaaccagtactagcagtggaggactctttggaaccacaaataccacctctaatccttttggcagcacatctggctccctctttgggccaagtagttttacagctgctcctactgggactactattaaatttaaccctccaactggtacagatactatggtcaaagctggagttagcactaacataagtaccaagcaccagtgtattactgctatgaaagaatatgaaagcaagtcactagaggaacttcgtttagaggattatcaggctaacaggaagggcccacagaaccaggtgggagcaggtaccacaactggcttgtttgggtcttctccagccacttccagcgcaacaggactcttcagctcctccaccactaattcaggctttgcatatggtcagaacaaaactgcctttggaactagtacaactggatttggaacaaatccaggtggtctctttggccaacagaatcagcagactaccagcctcttcagcaaaccatttggccaggctacaaccacccagaacactggcttttcctttggtaataccagcaccataggacagccaagcaccaacactatgggattatttggagtaacccaagcctcacagcctggaggtctttttgggacagctacaaacaccagcactgggacagcatttggaacaggaacaggtctctttgggcagaccaatactggatttggtgctgttggttcgaccctgtttggcaataacaagcttactacatttggaagcagcacaaccagtgcaccttcatttggtacaaccagtggcgggctctttggttttggcacaaataccagtgggaatagtatttttggaagtaaaccagcacctgggactcttggaactgggcttggtgcaggatttggaacagctcttggtgctggacaggcatctttgtttgggaacaaccaacctaagattggagggcctcttggtacaggagcctttggggcccctggatttaatactacgacagccactttgggctttggagccccccaggccccagtagctttgacagatccaaatgcttctgctgcccagcaggctgttctccagcagcacatcaatagtctaacatactcaccttttggagactctcctctcttccggaatccgatgtcagaccctaagaagaaggaagaggaaggcggtagcggtggcagcggtggtagcggcggctcctaatctaga

SEQ ID NO: 26 - protein, 799 aa, Artificial Sequence
  Synthesized
MKVSKGEELFTGVVPILVELDGDVNGHKFSVRGEGEGDATNGKLTLKFICTTGKLPVPWPTLVTTLTYGVQCFSRYPDYMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITLGMDELYKTMKGGSGGSGGSGGSLEMFNKSFGTPFGGGTGGFGTTSTFGQNTGFGTTSGGAFGTSAFGSSNNTGGLFGNSQTKPGGLFGTSSFSQPATSTSTGFGFGTSTGTANTLFGTASTGTSLFSSQNNAFAQNKPTGFGNFGTSTSSGGLFGTTNTTSNPFGSTSGSLFGPSSFTAAPTGTTIKFNPPTGTDTMVKAGVSTNISTKHQCITAMKEYESKSLEELRLEDYQANRKGPQNQVGAGTTTGLFGSSPATSSATGLFSSSTTNSGFAYGQNKTAFGTSTTGFGTNPGGLFGQQNQQTTSLFSKPFGQATTTQNTGFSFGNTSTIGQPSTNTMGLFGVTQASQPGGLFGTATNTSTGTAFGTGTGLFGQTNTGFGAVGSTLFGNNKLTTFGSSTTSAPSFGTTSGGLFGFGTNTSGNSIFGSKPAPGTLGTGLGAGFGTALGAGQASLFGNNQPKIGGPLGTGAFGAPGFNTTTATLGFGAPQAPVALTDPNASAAQQAVLQQHINSLTYSPFGDSPLFRNPMSDPKKKEEEGGSGGSGGSGGSSQETFSDLWKLLPEN

SEQ ID NO: 27 - DNA, 2414 bp, Artificial Sequence
  Synthesized
gcggccgcatgaaagtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgactacatgaagcagcacgacttcttcaagtccgccatgcccgaaggctacgtccaggagcgcaccatctccttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcacggccgacaagcagaagaacggcatcaaggcgaacttcaagatccgccacaacgtcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagaccatgaaaggcggtagcggtggcagcggtggtagcggcggctccctcgagatgtttaacaaatcatttggaacaccctttgggggtggcacaggtggctttggcacaacttcaacatttggacagaatactggctttggcactactagtggaggggcatttggaacatctgcatttggttctagcaacaatactggaggcctctttggaaattcacagactaaaccaggaggattgtttggaaccagttcatttagccagccagctacctccacaagcactggctttgggtttggtacgtcaacaggaacagcaaataccttgtttggaactgcaagcacagggaccagtctcttctcatcccaaaacaatgcctttgcacaaaataaaccaactggctttggcaattttggaaccagtactagcagtggaggactctttggaaccacaaataccacctctaatccttttggcagcacatctggctccctctttgggccaagtagttttacagctgctcctactgggactactattaaatttaaccctccaactggtacagatactatggtcaaagctggagttagcactaacataagtaccaagcaccagtgtattactgctatgaaagaatatgaaagcaagtcactagaggaacttcgtttagaggattatcaggctaacaggaagggcccacagaaccaggtgggagcaggtaccacaactggcttgtttgggtcttctccagccacttccagcgcaacaggactcttcagctcctccaccactaattcaggctttgcatatggtcagaacaaaactgcctttggaactagtacaactggatttggaacaaatccaggtggtctctttggccaacagaatcagcagactaccagcctcttcagcaaaccatttggccaggctacaaccacccagaacactggcttttcctttggtaataccagcaccataggacagccaagcaccaacactatgggattatttggagtaacccaagcctcacagcctggaggtctttttgggacagctacaaacaccagcactgggacagcatttggaacaggaacaggtctctttgggcagaccaatactggatttggtgctgttggttcgaccctgtttggcaataacaagcttactacatttggaagcagcacaaccagtgcaccttcatttggtacaaccagtggcgggctctttggttttggcacaaataccagtgggaatagtatttttggaagtaaaccagcacctgggactcttggaactgggcttggtgcaggatttggaacagctcttggtgctggacaggcatctttgtttgggaacaaccaacctaagattggagggcctcttggtacaggagcctttggggcccctggatttaatactacgacagccactttgggctttggagccccccaggccccagtagctttgacagatccaaatgcttctgctgcccagcaggctgttctccagcagcacatcaatagtctaacatactcaccttttggagactctcctctcttccggaatccgatgtcagaccctaagaagaaggaagaggaaggcggtagcggtggcagcggtggtagcggcggctccagccaggaaacctttagcgatctgtggaaactgctgccggaaaactaatctaga

SEQ ID NO: 28 - protein, 256 aa, Artificial Sequence
  Synthesized
MKVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKTMKGGSGGSGGSGGSLEHA

SEQ ID NO: 29 - DNA, 785 bp, Artificial Sequence
  Synthesized
gcggccgcatgaaagtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgtggcaccaacttcccctccgacggccccgtaatgcagaagaagacaatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaccatgaaaggcggtagcggtggcagcggtggtagcggcggctccctcgagcatgcataatctaga

SEQ ID NO: 30 - DNA, 324 bp, Artificial Sequence
  Synthesized
ctcgagatgactgatggtgctgtaaccaccagccagattccggcgagcgaacaggaaaccctggtgcgcccgaaaccgctgctgctgaaactgctgaaaagcgtgggcgcgcagaaagatacctataccatgaaagaagtgctgttttatctgggccagtatattatgaccaaacgcctgtatgatgaaaaacagcagcatattgtgtattgcagcaacgatctgctgggcgatctgtttggcgtgccgagctttagcgtgaaagaacatcgcaaaatttataccatgatttatcgcaacctggtggtggtgaactaatctaga